(19) United States
(12) Patent Application Publication
(10) Pub. No.: US 2024/0132440 A1
(43) Pub. Date: Apr. 25, 2024

(54) METHOD FOR THE PRODUCTION OF D-ERYTHRO-SPHINGOSINE AND ANALOGS THEREOF

(71) Applicant: CarboCode S.A., Cantanhede (PT)

(72) Inventors: Katharina Kettelhoit, Cantanhede (PT); Gyula Dekany, Cantanhede (PT); Györgyi Osztrovszky, Cantanhede (PT); Markus Schoewe, Cantanhede (PT); Karoly Agoston, Cantanhede (PT); Ricardo Figueiredo, Cantanhede (PT); Jorge Santos, Cantanhede (PT); Fabio Pereira, Cantanhede (PT); Ferenc Horvath, Cantanhede (PT)

(21) Appl. No.: 18/262,913

(22) PCT Filed: Jan. 25, 2022
(86) PCT No.: PCT/PT2022/050003

§ 371 Date: Jul. 25, 2023

(30) Foreign Application Priority Data
Jan. 25, 2021 (CH) 00066/21
Jan. 25, 2021 (CH) 00068/21

(51) Int. Cl. C07C 213/08; C07C 213/10; C07C 215/24; C07C 249/02; C07C 251/24; C07D 207/325; C07D 265/06; C07D 319/06; C07D 498/04; C07D 498/18; C12P 13/00

(52) U.S. Cl. CPC C07C 213/08; C07C 213/10; C07C 215/24; C07C 249/02; C07C 251/24; C07D 207/325; C07D 265/06; C07D 319/06; C07D 498/04; C07D 498/18; C12P 13/001

(57) ABSTRACT

The present invention relates to a method for the production of d-erythro-sphingosine and analogs thereof, wherein the method comprises a step of condensing a compound of formula (2):

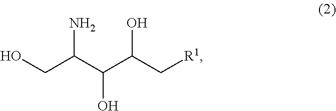

or a salt thereof,
   wherein
R¹ is hydrogen, a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of an alkoxy group, a secondary, or tertiary amine, a thioether, an acyloxy group, an acylamido group, a phosphorus containing functional group, a carboxyl group, or a carbonyl group,
with a compound of formula (3):

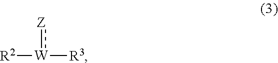

wherein
the bond [symbol] represents a double or a single bond,
W is C, or C(OR⁴),
Z is O, or OR⁵,
provided that:
when W is C, the bond [symbol] is a double bond and Z is O, or
when W is C(OR⁴), the bond [symbol] is a single bond and Z is OR⁵, and
   wherein
R² and R³ are independently selected from a saturated or unsaturated C₁₋₆ alkyl, a saturated or unsaturated cycloalkyl, or an aryl, each of which may be substituted or unsubstituted, or wherein one of R² and R³ is hydrogen, and the other rest is a saturated or unsaturated C₁₋₆ alkyl, a saturated or unsaturated cycloalkyl, or an aryl, each of which may be substituted or unsubstituted, or wherein R² and R³ may form a cyclic structure,
R⁴ and R⁵ are independently selected from a C₁₋₆ alkyl, a cycloalkyl, or an aryl, each of which may be substituted or unsubstituted.

### CROSS-REFERENCE TO RELATED APPLICATIONS

This application is a US national stage entry of PCT/PT2022/050003, filed on Jan. 25, 2022, which claims priority to and benefit of Switzerland Application No. 00066/21, filed on Jan. 25, 2021, and Switzerland Application No. 00068/21, filed on Jan. 25, 2021, the contents of which are incorporated herein by reference in their entireties.

The present invention relates to a novel and efficient method for the production of D-erythro-sphingosine or analogs thereof.

### BACKGROUND

Sphingoid bases are an important class of naturally occurring long-chain amino bases found in mammalian cells. Among naturally occurring sphingoid bases, D-erythro-sphingosine (CAS n°: 123-78-4), D-ribo-phytosphingosine (CAS n°: 388566-94-7), DL-erythro-dihydrosphingosine (CAS n°: 3102-56-5) and 6-hydroxy-D-erythro-sphingosine (CAS n°: 566203-07-4) are the most important.

Sphingoid bases, such as Derythro-sphingosine, constitute the backbone of sphingolipids such as ceramides, as well as phosphosphingolipids, phosphoceramides, and glycosphingolipids. Sphingolipids are an important class of polar lipids mainly found on the surface of eukaryotic cells. Sphingolipids are involved in diverse biological processes and play important structural and functional roles such as cell—cell recognition, communication, and intercellular adhesion. Particularly, GSLs such as gangliosides are found in the brain and play roles in neurological diseases, whereas ceramides are the main constituent of the stratum corneum lipid layer and have a major role in the water-retaining properties of the epidermis, as well as in the barrier function of the skin.

Accordingly, sphingolipids hold great potential as therapeutics, cosmetics, and as tools for the study of important biological processes. However, they are not readily available for fundamental and clinical research. In fact, sphingolipids such as ceramides and GSLs are characterized by a high structural complexity and their preparation represents a challenge.

A key step for the successful synthesis of natural and non-natural sphingolipids is the synthetic access to sphingoid bases.

Processes for the production of sphingoid bases, that are based on chemical synthesis, extraction from natural sources, or biotechnological methods have been reported.

For instance, a biotechnological method describing microbial strains capable of producing D-erythro-sphingosine have been described (EP1767644 A1). Limitations connected to this approach include poor conversion rates, difficult scale-up and purification.

Alternatively, D-erythro-sphingosine may be obtained by extraction from natural sources, such as for instance animal tissues (EP 3095451 A1, U.S. Pat. No. 5,532,141 A). However, this approach possesses several limitations including low yields, and the isolation of heterogeneous mixtures of structurally different compounds, which are potentially unsafe due to the possible presence of hazardous biological contaminants.

Attempts have been made to develop synthetic pathways for producing D-erythro-sphingosine and its analogs (R. J. B. H. N. van den Berg et al. J. Org. Chem. 2004, 69, 5699-5704; S. Kim et. al. J. Org. Chem. 2006, 71, 8661-8664; WO 2019238970 A1; U.S. Pat. No. 4,952,683 A; US 2003171621 A1; WO 1989012632 A1). Drawbacks connected to these methods comprise the numerous reaction steps, the use of expensive reagents as well as extreme reaction conditions.

To date, the existing isolation, biotechnological and synthetic methods have not been effective in producing large volumes of D-erythro-sphingosine.

Therefore, there is a demand for the development of novel synthetic methodologies which enable efficient and large-scale production of D-erythro-sphingosine characterized by high technological feasibilities and low costs.

### SUMMARY OF THE INVENTION

The present invention, in a first aspect, relates to a method for producing a sphingoid base of formula (1):

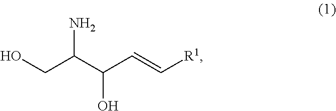

or a salt thereof,
from a compound of formula (2)

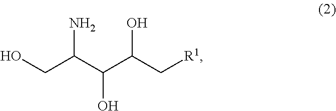

or a salt thereof,
   wherein
R1 is hydrogen, a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of an alkoxy group, a secondary, or tertiary amine, a thioether, an acyloxy group, an acylamido group, a phosphorus containing functional group, a carboxyl group, or a carbonyl group,

comprising a step of subjecting the compound of formula (2) to a condensation reaction with a compound of formula (3):

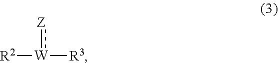

wherein
the bond [symbol] represents a double or a single bond,
W is C, or C(OR⁴),
Z is O, or OR⁵,
provided that:
when W is C, the bond [symbol] is a double bond and Z is O, or
when W is C(OR⁴), the bond [symbol] is a single bond and Z is OR⁵, and
   wherein
R² and R³ are independently selected from a saturated or unsaturated C₁₋₆ alkyl, a saturated or unsaturated cycloalkyl, or an aryl, each of which may be substituted or unsubstituted, or wherein one of R² and R³ is hydrogen, and the other rest is a saturated or unsaturated C₁₋₆ alkyl, a saturated or unsaturated cycloalkyl, or an aryl, each of which may be substituted or unsubstituted, or wherein R² and R³ may form a cyclic structure,
R⁴ and R⁵ are independently selected from a C₁₋₆ alkyl, a cycloalkyl, or an aryl, each of which may be substituted or unsubstituted.

In a second aspect, the present invention relates to a protected derivatives of a compound of formula (2), wherein the protected derivative of the compound of formula (2) is selected from the group consisting of compounds of formulas (4) to (11), or salts thereof:

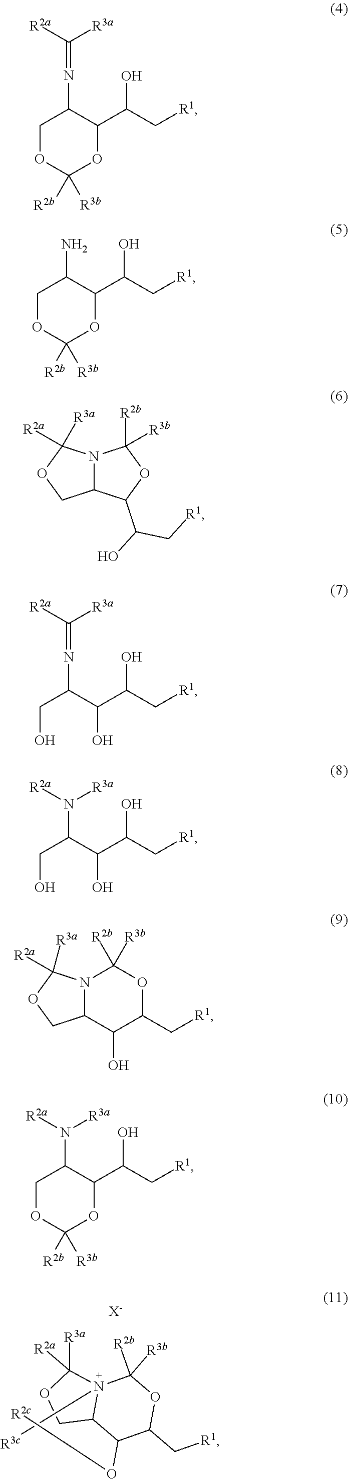

wherein
R¹, is as defined as for the compound of formula (2),
R^{2a}, R^{3a}, R^{2b}, R^{3b}, R^{2c} and R^{3c} are independently selected from a saturated or unsaturated C₁₋₆ alkyl, a saturated or unsaturated cycloalkyl, or an aryl, each of which may be substituted or unsubstituted, or wherein one of R^{2a} and R^{3a}, and/or one of R^{2b} and R^{3b}, and/or one of R^{2c} and R^{3c} is hydrogen and the other rest is a saturated or unsaturated C₁₋₆ alkyl, a saturated or unsaturated cycloalkyl, or an aryl, each of which may be substituted or unsubstituted, or wherein R^{2a} and R^{3a}, and/or R^{2b} and R^{3b}, and/or R^{2c} and R^{3c} may form a cyclic structure,
X⁻ is an organic or inorganic anion selected from chloride, perchlorate, sulfate, phosphate, polyphosphate, carboxylate, camphorsulfonate, or sulfonate.

In a third aspect, the present invention relates to a method for producing a sphingoid base of formula (1), wherein the method further comprising a step of introducing a leaving group at the C-4 position of the compound of formula (4), (5), (6), or (10) by substitution or replacement of the C-4 hydroxyl group, thereby obtaining a compound of formula (14), (15), (16), or (17) respectively, or salts thereof:

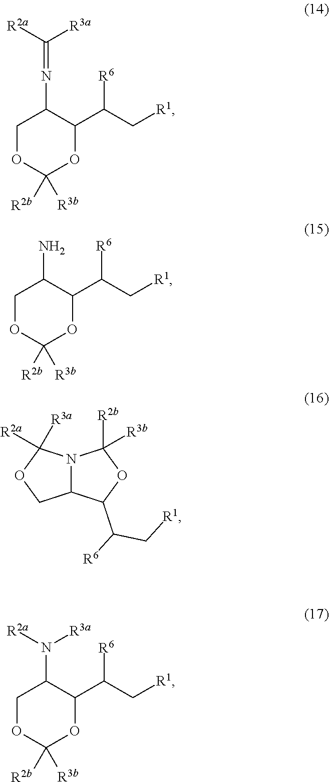

wherein
R¹, R^{2a}, R^{3a}, R^{2b}, and R^{3b} are as defined as for the compounds of formula (4), (5), (6), or (10),
R⁶ is a leaving group selected from a halide, a sulfonate, or a phosphite.

In a fourth aspect, the present invention relates to a method for producing a sphingoid base of formula (1), wherein the method further comprising a step of reacting the compound of formula (14), (15), (16), or (17) with a base thereby inducing an elimination reaction, and thereby obtaining a compound of formula (18), (19), (20) or (21), respectively, or salts thereof:

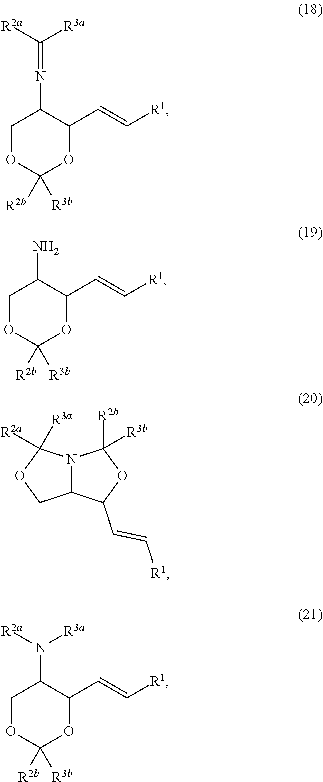

wherein
R¹, R^{2a}, R^{3a}, R^{2b}, and R^{3b} are as defined as for the compounds of formula (14), (15), (16), or (17).

In a fifth aspect, the present invention relates to a method for producing a sphingoid base of formula (1), wherein the method further comprising a step of subjecting a compound of formula (18), (19), (20) or (21) to acidic treatment thereby producing a sphingoid base of formula (1), or a salt thereof.

In a sixth aspect, the present invention relates to a method for producing a sphingoid base of formula (1) from a compound of formula (2), wherein the method further comprising steps of obtaining a compound of formula (2).

In a seventh aspect, the compound of formula (2) is obtained via the steps of:
fermenting at least one acetylated analog of the compound of formula (2) in a microorganism, preferably in a yeast cell;
separating the at least one acetylated analog of the compound of formula (2) from the whole fermentation material or the microbial biomass by using an organic solvent or super-critical CO₂ extraction;
subjecting the at least one acetylated analog of the compound of formula (2) to hydrolysis thereby producing the compound of formula (2).

### DETAILED DESCRIPTION OF THE INVENTION

The present inventors have established an economically feasible method for the production of D-erythro-sphingosine and analogs thereof. Advantages connected to this method include the production of structurally defined products in high purities, the absence of biohazardous contaminants, scalability, and reliability. Particularly, the method is characterized by high yields, the use of inexpensive and non-toxic reagents, and mild reaction conditions. Therefore, the method is suited for the large-scale production of highly pure D-erythro-sphingosine and analogs thereof, from a compound of formula (2):

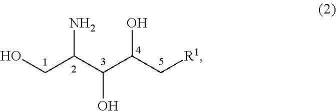

or a salt thereof,
   wherein
R¹ is hydrogen, a C₁₋₅₀ alkyl, preferably a C₁₋₁₅ alkyl, more preferably a C₁₀₋₁₅ C₁₋₁₅ alkyl, which may be saturated or contain one or more double and/or triple bonds, and/or which may contain one or more functional groups, the functional group being preferably selected from the group consisting of an alkoxy group, a secondary, or tertiary amine, a thioether, an acyloxy group, an acylamido group, a phosphorus containing functional group, a carboxyl group, or a carbonyl group,

the method comprising a step of subjecting the compound of formula (2) to a condensation reaction with a compound of formula (3):

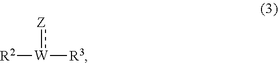

wherein
the bond [symbol] represents a double or a single bond,
W is C, or C(OR⁴),
Z is O, or OR⁵,
provided that:
when W is C, the bond [symbol] is a double bond and Z is O, or
when W is C(OR⁴), the bond [symbol] is a single bond and Z is OR⁵, and
   wherein
R² and R³ are independently selected from a saturated or unsaturated C₁₋₆ alkyl, a saturated or unsaturated cycloalkyl, or an aryl, each of which may be substituted or unsubstituted, or wherein at least one of R² and R³ is hydrogen, and the other rest is a saturated or unsaturated C₁₋₆ alkyl, a saturated or unsaturated cycloalkyl, or an aryl, each of which may be substituted or unsubstituted, or wherein R² and R³ may form a cyclic structure,
R⁴ and R⁵ are independently selected from a C₁₋₆ alkyl, a cycloalkyl, or an aryl, each of which may be substituted or unsubstituted.

Non-limiting embodiments of different aspects of the invention are described below and illustrated by non-limiting examples.

The terms, definitions and embodiments described throughout specification of the invention relate to all aspects and embodiments of the invention.

The term “a” grammatically is a singular, but it may as well mean the plural of e.g., the intended compound. For example, a skilled person would understand that in the expression “a compound of formula (2)”, the provision of not only one single a compound of formula (2), but of a variety of compounds of the same type is meant.

As used herein, the various functional groups or substituents represented will be understood to have a point of attachment at the functional group or atom having the dash (—). For example, in the case of —OTf it will be understood that the point of attachment is the oxygen atom. If a group is listed without a dash, then the attachment point is indicated by the plain and ordinary meaning of the recited group.

As used herein, the letters C and 0 refer to a carbon atom and an oxygen atom respectively.

The skilled person would understand that when speaking of position C-1, C-2, C-3, C-4, C-5 etc., reference is herein always made to the respective carbon atoms of sphingoid base of formula (1), or the compound of formula (2).

As used herein, the term “alkyl” refers to an acyclic straight or branched hydrocarbyl group having 1-50 carbon atoms which may be saturated or contain one or more double and/or triple bonds, so forming, for example, an alkenyl or an alkynyl, and/or which may be substituted or unsubstituted, as herein further described. Examples of “alkyl” include, but are not limited to, methyl, ethyl, n-propyl, isopropyl, isobutyl, n-butyl, sec-butyl, tert-butyl, isopentyl, n-pentyl, neo-pentyl, n-hexyl, ethenyl, propenyl, 1-butenyl, 2-butenyl, isobutenyl,1-pentenyl, 2-pentenyl, 2-methyl-1-butenyl, 3-methyl-1-butenyl, 2-methyl-2-butenyl, 1-hexenyl, 2-hexenyl, 3-hexenyl, methylpentenyl, dimethylbutenyl, ethynyl, propynyl, 1-butynyl, 2-butynyl, pentynyl, and hexynyl, each of which may be substituted or unsubstituted. Typically, the term alkyl refers to a straight saturated acyclic hydrocarbyl group having 1-15 carbons, which may be substituted or unsubstituted.

As used herein, the term “cycloalkyl” refers to a non-aromatic cyclic hydrocarbyl group having 3-12 ring carbon atoms, which may be mono- or polycyclic, which may contain fused rings, preferably 1 to 3 fused or unfused rings, which may be saturated or contain one or more double bonds (so, forming for example a cycloalkenyl), and/or which may be substituted or unsubstituted, as herein further described. Examples of cycloalkyls include, but are not limited to, cyclopropane, cyclobutane, cyclopentane, cyclohexane, cycloheptane, cyclooctane, cyclopropene cyclobutene, cyclopentene, cyclohexene, cycloheptene, and cyclooctene, each of which may be substituted or unsubstituted. Typically, the term “cycloalkyl” refers to a non-aromatic monocyclic hydrocarbyl group having 3-6 carbon atoms, which may be substituted or unsubstituted.

As used herein, the term “aryl” refers to an aromatic cyclic hydrocarbyl group having 5-14 ring carbon atoms, which may be mono- or polycyclic, which may contain fused rings, preferably 1 to 3 fused or unfused rings, and which may contain one or more heteroatoms, and/or which may be substituted or unsubstituted, as herein further described. Examples of “aryl” include, but are not limited to, phenyl, naphtyl, anthracyl, phenantryl, pyrrolyl, imidazolyl, thiophenyl, furanyl, oxazolyl, thiazolyl, pyridinyl, pyrimidinyl, pyrazinyl, triazinyl, and benzofuranyl, each of which may be substitute or unsubstituted. Typically, the term “aryl” refers to a substituted or unsubstituted phenyl.

As used herein, the term “substituted” means that the group in question is substituted with a group which typically modifies the general chemical characteristics of the group in question. The substituents can be used to modify characteristics of the molecule, such as molecule stability, molecule solubility and the ability of the molecule to form crystals. The person skilled in the art will be aware of other suitable substituents of a similar size and charge characteristics, which could be used as alternatives in a given situation.

In connection with the terms “alkyl”, “cycloalkyl” and “aryl” the term “substituted” means that the group in question is substituted one or several times, preferably 1 to 3 times, with group(s) selected from hydroxy (which when bound to an unsaturated carbon atom may be present in the tautomeric keto form), C₁₋₆-alkoxy (i.e. C1-6-alkyl-oxy), C₂₋₆-alkenyloxy, carboxy, oxo, C₁₋₆-alkoxycarbonyl, C₁₋₆-alkylcarbonyl, formyl, aryl, aryloxycarbonyl, aryloxy, arylamino, arylcarbonyl, heteroaryl, heteroarylamino, heteroaryloxycarbonyl, heteroaryloxy, heteroarylcarbonyl, amino, mono- and di(C₁₋₆-alkyl)amino, carbamoyl, mono- and di-C₁₋₆-alkyl-aminocarbonyl, amino-C₁₋₆-alkyl-aminocarbonyl, mono- and di-C₁₋₆-alkyl-amino-C₁₋₆-alkyl-aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, halogen, where any alkyl, alkoxy, and the like representing substituents may be substituted with hydroxy, C₁₋₆- alkoxy, C₂₋₆-alkenyloxy, carboxy, C₁₋₆-alkylcarbonylamino, halogen, C₁₋₆-alkylthio, C₁₋₆-alkyl-sulphonyl-amino, or guanidino.

As used herein, when referring to a substituted aryl group, the relative position occupied by a substituent on the aromatic ring is typically indicated by the prefixes o-, m-, and p-, wherein the prefix o- refers to an ortho-substitution, the prefix m- refers to a metha-substitution, and the prefix p- refers to a para-substitution. Ortho-, meta-, and para-substitution may also be indicated by the prefixes 2-, 3- and 4-, respectively. In the context of the present invention, the prefixes o-, m-, and p-, and the prefixes 2-, 3- and 4- may be used interchangeably.

The term “protected derivative” refers to a modified form of a compound containing one or more protecting groups.

The term “protecting group” refers to a group which has been introduced onto a functional group in a compound, and which modifies the chemical reactivity of said functional group. Typically, the protecting group modifies the chemical reactivity of the functional group in such a way that it renders said functional group chemically inert to the reaction conditions used when a subsequent chemical transformation is performed on said compound.

The person skilled in the art would understand that a protecting group is introduced onto a functional group of a compound through the reaction between the (unprotected) functional group and a protecting group precursor, therefore generating a “protected” derivative of said compound.

As used herein, the term “leaving group” means an atom or a group (which may be charged or uncharged) that becomes detached from an atom belonging to the residual or main part of the molecule taking part in a specific reaction, such as for example a nucleophilic substitution or an elimination reaction. Example of leaving groups include, but are not limited to, halides triflates (—OTf), diazonium salts (N₂⁺), mesylates (—OMs), tosylates (—OTs), nosylates (—ONs), brosilate, imidazole-1-sulfonate (—OSO₂Im), 2-methylimidazole-1-sulfonate, or dichlorophosphite (—OPCl₂), and the like.

The term “cyclic structures” refers to a carbocycle ring, wherein all the ring atoms are carbons, or to a heterocycle ring, wherein one or more carbon atoms are replaced by an oxygen atom, a nitrogen atom and/or a sulfur atom. The carbocycle or the heterocycle cyclic structures are characterized by 5 to 8 ring atoms, preferably 5 to 6 ring atoms, may be saturated or contain double bonds, may be non-aromatic or aromatic and may be unsubstituted or substituted. Example of cyclic structures include, but are not limited to, cyclopentane, cyclohexane, piperidine and pyrrolidine, and the like.

As used herein, the term “aprotic solvent” refers to any solvent which lacks a labile (acidic) hydrogen atom. The aprotic solvent may be a polar aprotic solvent, or a non-polar solvent. Polar aprotic solvents are characterized by a net positive dipole moment, and a relatively high dielectric constant. Examples of polar aprotic solvents include, but are not limited to, hydrofurans (e.g. tetrahydrofuran, etc.), hydropyrans, organic esters (e.g. ethylacetate, propylacetate, butyl acetate, etc.), ketones (e.g. acetone, methyl-ethyl ketone, methyl-isobutyl ketone, etc.), dimethylformamide, acetonitrile, propionitrile, dimethylsulfoxide, propylene carbonate, N-methyl-2-pyrrolidone, and the like. Non-polar solvents are characterized by a low dielectric constant and are not miscible with water. Examples of non-polar solvents include, but are not limited to alkane (e.g. hexane, heptane, cyclohexane, etc.), aromatic hydrocarbons (e.g. toluene, xylene, mesitylene etc.) ethers (e.g. dioxane, methyl-tertbutyl ether, diisopropyl ether, etc.), and the like.

As used herein, the term “rest” refers to a variable atom or a variable functional group of a compound and it is represented with a R. The rest may be independently selected from a set of atoms or functional groups, or the selection of one rest may depend on the selection of another rest. For example, for a compound of formula (3) the rests R² and R³, may be independently selected, or the selection of R² may depend on the selection of R³. Therefore, the expression “one of R² and R³, is hydrogen and the other rest is an alkyl” means that when R² is hydrogen the other rest R³ is an alkyl, or that when R² is an alkyl the other rest R³ is hydrogen.

In the context of the present invention, the terms “about”, “around”, or “approximate” are applied interchangeably to a particular value (e.g. “a temperature of about 25° C.”, “a temperature of around 25° C.”, or “a temperature of approximate 25° C.”), or to a range (e.g. “an amount from about 1% to about 99%”, “an amount from around 1% to around 99%”, or “an amount from approximate 1% to 30 approximate 99%”) , to indicate a deviation from 0.1% to 10% of that particular value or range.

As used herein, the term “condensation reaction” refers to a chemical reaction in which two organic compounds react to produce an addition product, and an elimination product such as water or an alcohol. The condensation reaction may be performed in the presence of an acid, a base, or a catalyst. Examples of organic compounds that can take part into a condensation reaction include, but are not limited to, alcohols, aldehydes, ketones, acetals, ketals, esters, alkynes (acetylenes), amines, and the like. Example of condensation reactions include, but are not limited to aldol condensation, Claisen condensation, Knoevenagel condensation, Dieckman condensation, and the like. Typically, in the context of the present invention, a condensation reaction refers to a chemical reaction, wherein the hydroxyl group(s) and the amino group(s) of an amino alcohol, such as the compound of formula (2), react with an aldehyde, a ketone, an acetal, or a ketal, such as a compound of formula (3), (12), or (13), or a combination thereof to form an addition product such as a compound of formula (4), (5), (6), (7), (8), (9), (10), or (11), and an elimination product such as water or an alcohol.

As used herein, the term “acetylated analog of the compound of formula (2)” refers to an analog of the compound of formula (2), wherein the C-1 hydroxyl group, and/or the C-3 hydroxyl group, and/or the C-4 hydroxyl group, and/or the C-2 amino group are acetylated. Accordingly, the acetylated analog of the compound of formula (2) may be a mono-, di-, tri-, tetra-acetylated analog of the compound of formula (2), or the acetylated analog of the compound of formula (2) is a mixture of mono-, di-, tri, and tetra-acetylated analogs of the compound of formula (2).

In the context of the present invention, the terms “analog” and “derivative” may be used interchangeably to describe a compound which differ from an original compound in that, one or more structural components of the original compound, such as one or more atoms, functional groups, or substructures, are replaced with other atoms, groups, or substructures.

The present invention discloses a method for the synthesis of a sphingoid base of formula (1):

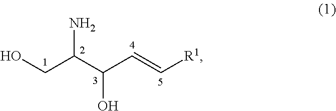

or a salt thereof,
from a compound of formula (2)

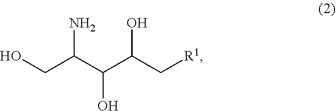

or a salt thereof,
   wherein

R¹ is hydrogen, a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of an alkoxy group, a secondary, or tertiary amine, a thioether, an acyloxy group, an acylamido group, a phosphorus containing functional group, a carboxyl group, or a carbonyl group.

In some embodiments, for the sphingoid base of formula (1) and for the compound of formula (2) R¹ is a substituted C₁₋₅₀ alkyl, preferably a C₁₋₁₅ alkyl, more preferably a C₁₀₋₁₅ alkyl, which may be saturated or contain one or more double and/or triple bonds, and wherein the substituent are selected from the group consisting of hydroxyl group, alkoxy group, a primary, a secondary, or a tertiary amine, a thiol, a thioether, an acyloxy group, an acylamido group, a phosphorus containing functional group, a carboxyl group, or a carbonyl group.

In some embodiments for the sphingoid base of formula (1) and for the compound of formula (2) R¹ is a linear saturated unsubstituted C₁₀₋₁₅ C₁₋₁₅ alkyl, preferably a C₁₃ alkyl. In some preferred embodiments, the sphingoid base of formula (1) and the compound of formula (2) correspond to D-erythro-sphingosine and D-ribo-phytosphingosine, respectively.

In some embodiments for the sphingoid base of formula (1) and for the compound of formula (2) R¹ is hydrogen. In some embodiments, the sphingoid base of formula (1) and the compound of formula (2) represent truncated D-erythro-sphingosine and truncated D-ribo-phytosphingosine, respectively.

The method for the synthesis of a sphingoid base of formula (1), disclosed by the present invention, comprises a step of subjecting the compound of formula (2) to a condensation reaction with a compound of formula (3).

In some embodiments, the compound of formula (3) is a compound of formula (12):

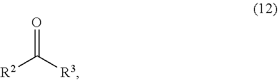

wherein
R² and R³ are independently selected from a saturated or unsaturated C₁₋₆ alkyl, a saturated or unsaturated cycloalkyl, or an aryl, each of which may be substituted or unsubstituted, or wherein at least one of R² and R³ is hydrogen, and the other rest is a saturated or unsaturated C₁₋₆ alkyl, a saturated or unsaturated cycloalkyl, or an aryl, each of which may be substituted or unsubstituted, or wherein R² and R³ may form a cyclic structure.

The compound of formula (12) may exist in the tautomeric form of an enol, also referred to as enolic form. Typically, an enol is formed via the migration of a hydrogen from the α-carbon to the oxygen of a carbonyl compound such as the compound of formula (12). The compound of formula (12) and its enolic form are typically in equilibrium, wherein the equilibrium may also be referred to as keto-enol equilibrium. Depending on the conditions the equilibrium may be shifted towards the keto form or the enol form of the compound of formula (12). However, it is to be understood, that the absolute structure of the compound of formula (12) is not critical to the concept of the present invention.

In some embodiments, the compound of formula (3) is a compound of formula (13):

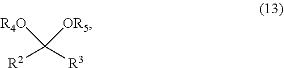

wherein
R² and R³ are independently selected from a saturated or unsaturated C₁₋₆ alkyl, a saturated or unsaturated cycloalkyl, or an aryl, each of which may be substituted or unsubstituted, or wherein one of R² and R³ is hydrogen, and the other rest is a saturated or unsaturated C₁₋₆ alkyl, a saturated or unsaturated cycloalkyl, or an aryl, each of which may be substituted or unsubstituted, or wherein R² and R³ may form a cyclic structure,
R⁴ and R⁵ are independently selected from a C₁₋₆ alkyl, or an aryl, each of which may be substituted or unsubstituted.

In some embodiments, for the compound of formula (12), R² and R³ are independently selected from a saturated or unsaturated C₁₋₆ alkyl, a saturated or unsaturated cycloalkyl, or an aryl, each of which may be substituted or unsubstituted, or R² and R³ may form a cyclic structure. Accordingly, in some embodiments, the compound of formula (12) is a ketone.

In some embodiments, for the compound of formula (12), R² and R³ are independently selected from methyl, ethyl, propyl, isopropyl, butyl, isobutyl, pentyl, isopentyl, hexyl, phenyl, or R² and R³ form a cyclohexyl, or a cyclopentyl.

In some embodiments the compound of formula (12) is a ketone selected from acetone, diethyl ketone, methyl isobutyl ketone, butan-2-one, cyclopentanone, cyclohexanone, hexane-2,5-dione, and acetophenone.

In some embodiments, for the compound of formula (12), one of R² and R³ is hydrogen, and the other rest is a saturated or unsaturated C₁₋₆ alkyl, a saturated or unsaturated cycloalkyl, or an aryl, each of which may be substituted or unsubstituted. Accordingly, in some embodiments, the compound of formula (12) is an aldehyde.

In some embodiments, for the compound of formula (12) one of R² and R³ is hydrogen, and the other rest is selected from methyl, ethyl, propyl, isopropyl, butyl, isobutyl, pentyl, isopentyl, phenyl, o-methoxyphenyl, m-methoxyphenyl, p-methoxyphenyl, o-methylphenyl, m-methylphenyl, p-methylphenyl, o-chlorophenyl, m-chlorophenyl, p-chlorophenyl, o-nitrophenyl, m-nitrophenyl, p-nitrophenyl, o-trifluoromethyl-phenyl, m-trifluoromethyl-phenyl, or p-trifluoromethyl-phenyl.

In some preferred embodiments, for the compound of formula (12) one of R² and R³ is hydrogen, and the other rest is selected from phenyl, p-methoxyphenyl, p-methylphenyl, or p-chlorophenyl.

In some embodiments, the compound of formula (12) is an aldehyde selected from acetaldehyde, 1-propanal, 2,3-(methylenedioxy)benzaldehyde, benzaldehyde, substituted benzaldehyde, preferably o-methoxybenzaldehyde, m-methoxybenzaldehyde, p-methoxybenzaldehyde, o-methylbenzaldehyde, m-methylbenzaldehyde, p-methylbenzaldehyde, o-chlorobenzaldehyde, m-chlorobenzaldehyde, p-chlorobenzaldehyde, o-nitro-benzhaldehyde, m-nitro-benzhaldehyde, p-nitro-benzhaldehyde, o-(trifluoromethyl)-benzhaldehyde, m-(trifluoromethyl)-benzhaldehyde, and p-(trifluoromethyl)benzaldehyde.

In some preferred embodiments, the compound of formula (12) is an aldehyde selected from benzaldehyde, p-methoxybenzaldehyde, p-methylbenzaldehyde, and p-chlorobenzaldehyde.

In some embodiments, for the compound of formula (13) R² and R³ are independently selected from a saturated or unsaturated C₁₋₆ alkyl, a saturated or unsaturated cycloalkyl, or an aryl, each of which may be substituted or unsubstituted, or R² and R³ may form a cyclic structure. Accordingly, in some embodiments, the compound of formula (13) is a ketal.

In some embodiments, for the compound of formula (13) R² and R³ are independently selected from methyl, ethyl, propyl, isopropyl, butyl, isobutyl, pentyl, isopentyl, hexyl, phenyl, or R² and R³ form a cyclohexyl, or a cyclopentyl.

In some embodiments, for the compound of formula (13) one of R² and R³ is hydrogen, and the other rest is a saturated or unsaturated C₁₋₆ alkyl, a saturated or unsaturated cycloalkyl, or an aryl, each of which may be substituted or unsubstituted. Accordingly, in some embodiments, the compound of formula (13) is an acetal.

In some embodiments, for the compound of formula (13) one of R² and R³ is hydrogen, and the other rest is selected from methyl, ethyl, propyl, isopropyl, butyl, isobutyl, pentyl, isopentyl, phenyl, o-methoxyphenyl, m-methoxyphenyl, p-methoxyphenyl, o-methylphenyl, m-methylphenyl, p-methylphenyl, o-chlorophenyl, m-chlorophenyl, p-chlorophenyl, o-nitrophenyl, m-nitrophenyl, p-nitrophenyl, o-trifluoromethyl-phenyl, m-trifluoromethyl-phenyl, or p-trifluoromethyl-phenyl.

In some embodiments, for the compound of formula (13) R⁴ and R⁵ are selected from methyl, ethyl, and phenyl.

In some preferred embodiments, for the compound of formula (13) one of R² and R³ is hydrogen, and the other rest is selected from phenyl, p-methoxyphenyl, p-methylphenyl, or p-chlorophenyl, and R⁴ and R⁵ are methyl. Accordingly in some preferred embodiments the compound of formula (13) is an acetal selected from benzaldehyde dimethyl acetal, anisaldehyde dimethyl acetal, p-methylbenzaldehyde dimethyl acetal, or p-chlorobenzaldehyde dimethyl acetal.

In some embodiments, the condensation reaction is performed by reacting the compound of formula (2) with a combination of compounds of formula (12) and/or (13). As described by the preceding embodiments, the compound of formula (12) represents a ketone or an aldehyde, and the compound of formula (13) represents an acetal or a ketal. Accordingly, in some embodiments, the condensation reaction is performed by reacting the compound of formula (2) with a combination of two types of ketones, or with a combination of two types of aldehydes, or with a combination of two types of acetals, or with a combination of two types of ketals, or with a combination of an acetal and a ketal, or with a combination of a ketone and an aldehyde, or with a combination of a ketone and an acetal.

The condensation reaction between the compound of formula (2) and the compound of formula (3), (12), or (13), or the combination thereof yields a protected derivative of the compound of formula (2).

In some embodiments the protected derivative of the compound of formula (2) is a compound of formula (4):

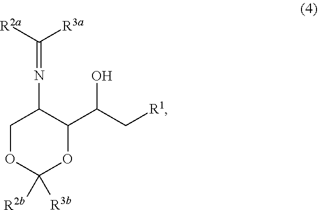

wherein
R¹, is as defined as for the compound of formula (2),
R^{2a}, R^{3a}, R^{2b}, and R^{3a} are independently selected from a saturated or unsaturated C₁₋₆ alkyl, a saturated or unsaturated cycloalkyl, or an aryl, each of which may be substituted or unsubstituted, or wherein one of R^{2a} and R^{3a}, and/or one of R^{2b} and R^{3b} is hydrogen and the other rest is a saturated or unsaturated C₁₋₆ alkyl, a saturated or unsaturated cycloalkyl, or an aryl, each of which may be substituted or unsubstituted, or wherein R^{2a} and R^{3a}, and/or R^{2b} and R^{3b} may form a cyclic structure.

The compound of formula (4) may exist in the tautomeric form of formula (22):

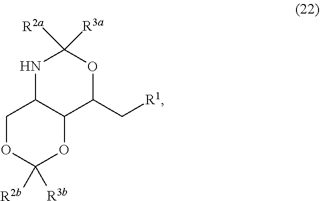

wherein
R¹, R^{2a}, R^{3a}, R^{2b}, and R^{3b} are as defined as for the compound of formula (4).

The compound of formula (4) and its tautomeric form of formula (22) are typically in equilibrium, wherein the compound of formula (4) represent the open-chain tautomer and the compound of formula (22) the cyclic tautomer. Depending on the conditions, the equilibrium between the two tautomeric forms may be shifted towards the open-chain tautomer of formula (4), or towards the cyclic tautomer of formula (22). Typically, the open-chain tautomer of formula (4) is favored in solution, whereas the cyclic tautomer of formula (22) is favored in the solid state. The equilibrium between the tautomeric forms may be represented as follow:

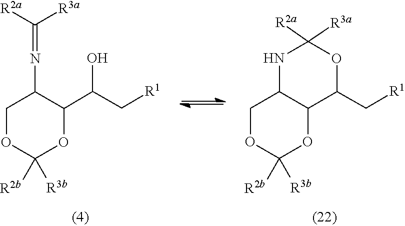

It is to be understood, however, that the absolute structure of the compound of formula (4) is not critical to the concept of the present invention.

In some embodiments, the protected derivative of the compound of formula (2) is a compound of formula (5), or a salt thereof:

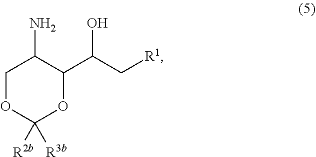

wherein
R¹, is as defined as for the compound of formula (2),
R^{2b}, and R^{3b} are independently selected from a saturated or unsaturated C₁₋₆ alkyl, a saturated or unsaturated cycloalkyl, or an aryl, each of which may be substituted or unsubstituted, or wherein one of R^{2b} and R^{3b} is hydrogen, and the other rest is a saturated or unsaturated C₁₋₆ alkyl, a saturated or unsaturated cycloalkyl, or an aryl, each of which may be substituted or unsubstituted, or wherein R^{2b} and R^{3b} may form a cyclic structure.

In some embodiments, the protected derivative of the compound of formula (2) is a compound of formula (6), or a salt thereof:

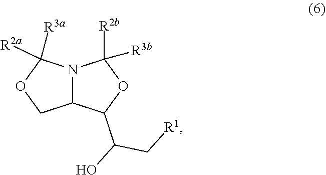

wherein
R¹, is as defined as for the compound of formula (2),

R^{2a}, R^{3a}, R^{2b}, and R^{3b} are independently selected from a saturated or unsaturated C₁₋₆ alkyl, a saturated or unsaturated cycloalkyl, or an aryl, each of which may be substituted or unsubstituted, or wherein one of R^{2a} and R^{3a}, and/or one of R^{2b} and R^{3b} is hydrogen and the other rest is a saturated or unsaturated C₁₋₆ alkyl, a saturated or unsaturated cycloalkyl, or an aryl, each of which may be substituted or unsubstituted, or wherein R^{2a} and R^{3a}, and/or R^{2b} and R^{3b} may form a cyclic structure.

In some embodiments, the protected derivative of the compound of formula (2) is a compound selected from the group consisting of compounds of formula (7) to (11), or a salt thereof:

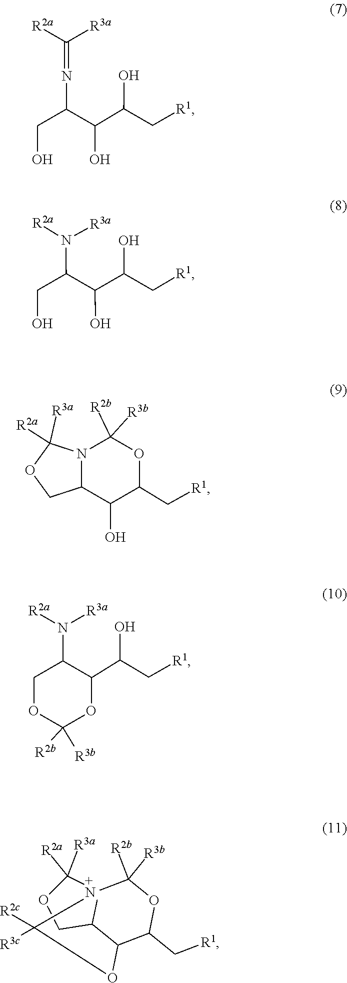

wherein
R¹, is as defined as for the compound of formula (2),
R^{2a}, R^{3a}, R^{2b}, R^{3b}, R^{2c} and R^{3c} are independently selected from a saturated or unsaturated C₁₋₆ alkyl, a saturated or unsaturated cycloalkyl, or an aryl, each of which may be substituted or unsubstituted, or wherein one of R^{2a} and R^{3a}, and/or one of R^{2b} and R^{3b}, and/or one of R^{2c} and R^{3c} is hydrogen and the other rest is a saturated or unsaturated C₁₋₆ alkyl, a saturated or unsaturated cycloalkyl, or an aryl, each of which may be substituted or unsubstituted, or wherein R^{2a} and R^{3a}, and/or R^{2b} and R^{3b}, and/or R^{2c} and R^{3c} may form a cyclic structure,
X⁻ is an organic or inorganic anion selected from chloride, perchlorate, sulfate, phosphate, polyphosphate, carboxylate, camphorsulfonate, or sulfonate.

The compound of formula (7) may exist in the tautomeric form of formula (23), or salts thereof:

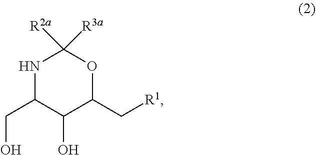

wherein
R¹, R^{2a} and R^{3a} are as defined as for the compound of formula (7).

The compound of formula (7) and its tautomeric form of formula (23) are typically in equilibrium, wherein the compound of formula (7) represent the open-chain tautomer and the compound of formula (23) the cyclic tautomer. Depending on the conditions, the equilibrium between the two tautomeric forms may be shifted towards the open-chain tautomer of formula (7), or towards the cyclic tautomer of formula (23). Typically, the open-chain tautomer of formula (7) is favored in solution, whereas the cyclic tautomer of formula (23) is favored in the solid state.

The equilibrium between the tautomeric forms may be represented as follow:

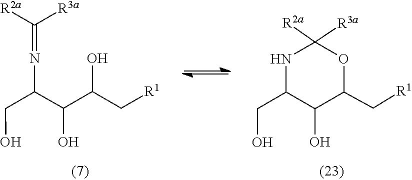

It is to be understood, however, that the absolute structure of the compound of formula (7) is not critical to the concept of the present invention.

In some embodiments, the compound of formula (10) is a compound of formula (24), or a salt thereof:

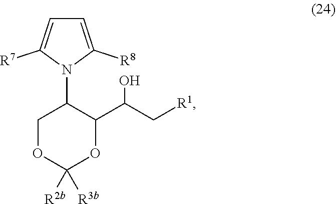

wherein
R¹, R^{2b}, and R^{3b} are as defined as for the compound of formula (10),

R⁷ and R⁸ are independently selected from hydrogen, a C₁₋₆ alkyl, which may be saturated or contain one or more double and/or triple bonds, and/or which may be substituted or unsubstituted.

In some preferred embodiments, for the compounds of formulas (4), (5), (6), (7), (8), (9), (10), (11), (22), (23), and (24) R¹ is a linear, unsubstituted, saturated C₁₃ alkyl, one of R^{2a} and R^{3a}, and one of R^{2b} and R^{3b} is hydrogen, and the other rest is a phenyl.

In some embodiments, for the compounds of formulas (4), (5), (6), (7), (8), (9), (10), (11), (22), (23), and (24) R¹ is a linear unsubstituted saturated C₁₃ alkyl, one of R^{2a} and R^{3a}, and one of R^{2b} and R^{3b} is hydrogen, and the other rest is p-methoxyphenyl.

In some embodiments, for the compounds of formulas (4), (5), (6), (7), (8), (9), (10), (11), (22), (23), and (24) R¹ is a linear unsubstituted saturated C₁₃ alkyl, one of R^{2a} and R^{3a}, and one of R^{2b} and R^{3b} is hydrogen, and the other rest is a is p-methylphenyl.

In some embodiments, for the compounds of formulas (4), (5), (6), (7), (8), (9), (10), (11), (22), (23), and (24) R¹ is a linear unsubstituted saturated C₁₃ alkyl, one of R^{2a} and R^{3a}, and one of R^{2b} and R^{3b} is hydrogen, and the other rest is a is p-chlorophenyl.

In some embodiments, for the compounds of formulas (4), (5), (6), (7), (8), (9), (10), (11), (22), (23), and (24) R¹ is a linear unsubstituted saturated C₁₃ alkyl, one of R^{2a} and R^{3a} is hydrogen, and the other rest is phenyl, and one of R^{2b} and R^{3b} is hydrogen and the other rest a p-methoxyphenyl.

In some embodiments, for the compounds of formulas (4), (5), (6), (7), (8), (9), (10), (11), (22), (23). and (24) R¹ is a linear unsubstituted saturated C₁₃ alkyl, one of R^{2a} and R^{3a} is hydrogen, and the other rest is p-methoxyphenyl, and one of R^{2b} and R^{3b} is hydrogen and the other rest a phenyl.

In some embodiments, for the compounds of formulas (4), (5), (6), (7), (8), (9), (10), (11), (22), (23), and (24) R¹ is a linear unsubstituted saturated C₁₃ alkyl, and R^{2a}, R^{3a}, R^{2b} and R^{3b} are methyl.

In some embodiments, for the compounds of formulas (4), (5), (6), (7), (8), (9), (10), (11), (22), (23), and (24) R¹ is a linear unsubstituted saturated C₁₃ alkyl, one of R^{2a} and R^{3a} is hydrogen, and the other rest is phenyl, and R^{2b} and R^{3b} are methyl.

In some embodiments, for the compounds of formulas (11), one of R^{2c} and R^{3c} is hydrogen, and the other rest is phenyl, p-methoxyphenyl, p-methylphenyl, p-chlorophenyl, preferably phenyl, and X⁻ is a tosylate (—OTs).

In some embodiments, the compound of formula (8), is a compound of formula (25), or a salt thereof:

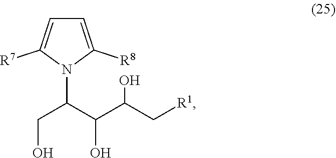

wherein R¹, R⁷, and R⁸ are as defined as for the compound of formula (24).

In some embodiments, for the compound of formula (25) R¹ is a linear unsubstituted saturated C₁₃ alkyl, and R⁷ and R⁸ are methyl.

In some embodiments, the protected derivative of the compound of formula (2) is a compound selected from the group consisting of compounds of formula (26) to (27):

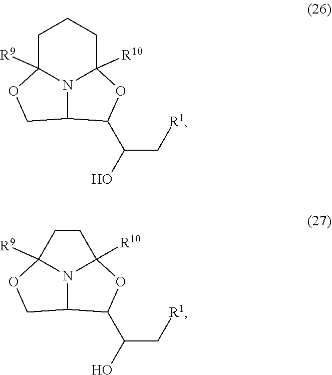

wherein
R¹ is as defined as for the compound of formula (2),
R⁹ and R¹⁰ are independently selected from a C₁₋₆ alkyl, which may be saturated or contain one or more double and/or triple bonds, and/or which may be substituted or unsubstituted.

In the context of the present invention compounds of formulas (4), (5), (6), (7), (8), (9), (10), (11), (22), (23), (24), (25), (26), or (27) represents the addition product of the condensation reaction between the compound of formula (2) and a compound of formula (3), (12), or (13), or a combination thereof.

The person skilled in the art, would understand that the addition products of formulas (4), (5), (6), (7), (8), (9), (10), (11), (22), (23), (24), (25), (26), or (27) may be obtained starting from the same reactants. For example, both compounds of formula (4) and (6) may be obtained by reacting the compound of formula (2) with a certain aldehyde, or a certain ketone, or a certain acetal, or a certain combination thereof. However, the selectivity of the reaction may be directed towards the formation of the compound of formula (4) rather than the compound of formula (6), or it may be directed towards the formation of the compound of formula (6) rather than the compound of formula (4), by the selection of a particular set of reaction conditions.

The reaction conditions that can direct the selectivity of the condensation reaction may comprise at least one of the following:
the use of variable ratios of the compound of formula (2) and of the compound of formula (3), (12), or (13), or the combination thereof, and/or
the use of an acid, and/or
the removal of water and/or alcohol formed during the condensation reaction, and/or
controlling the reaction kinetically, or
controlling the reaction thermodynamically.

Controlling the reaction kinetically, refers to a set of conditions used in a chemical reaction that typically enable the selective formation of the faster forming product, also referred to as the kinetic product. The conditions that typically enable the selective formation of the kinetic product, may also be referred to as kinetic conditions. Kinetic conditions may comprise the use of low temperatures and/or a short reaction time.

Controlling the reaction thermodynamically, refers to a set of conditions used in a chemical reaction that typically enable the selective formation of the more stable product, also referred to as the thermodynamic product. The conditions that typically enable the selective formation of the thermodynamic product, may also be referred to as thermodynamic conditions. Thermodynamic conditions may comprise the use of high temperatures and/or a long reaction time.

The compound of formula (3), (12), or (13), or the combination thereof may be used in variable amounts compared to the amount of the compound of formula (2). Typically, about 1 to about 4 molar equivalents of the compound of formula (3), (12), or (13) or the combination thereof is used, based on the amount of the compound of formula (2). Therefore, in some embodiments, the condensation reaction is performed by reacting the compound of formula (2) with about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 molar equivalents of the compound of formula (3), (12), or (13), or the combination thereof.

In some embodiments, about 2.0 molar equivalents of the compound of formula (3), (12), or (13), or the combination thereof are used based on the amount of the compound of formula (2).

In some embodiments, about 3.0 molar equivalents of the compound of formula (3), (12), or (13), or the combination thereof, are used based on the amount of the compound of formula (2).

In some embodiments, the about 2.0, molar equivalents of the compound of formula (3), (12), or (13), or the combination thereof, are added in one portion to the reaction.

In some embodiments, the about 3.0 molar equivalents of the compound of formula (3), (12), or (13), or the combination thereof, are added in one portion to the reaction.

In some embodiments, the about 2.0 molar equivalents of the compound of formula (3), (12), or (13), or the combination thereof, are added portion-wise to the reaction in two portions of about 1.0 molar equivalent each, based on the amount of the compound of formula (2), thereby producing an intermediate addition product.

In some embodiments, the about 3.0 molar equivalents of the compound of formula (3), (12), or (13), or the combination thereof, are added portion-wise to the reaction in two portions of about 1.5 molar equivalent each, based on the amount of the compound of formula (2), thereby producing an intermediate addition product.

The intermediate addition product may be isolated and subsequently reacted with the second portion of the compound of formula (3), (12), or (13), or the combination thereof. Accordingly, in some embodiments, compounds of formula (5), (7), (8), (23), or (25) represent the intermediate addition products of the portion-wise addition of an aldehyde, a ketone, an acetal, or a ketal.

In some embodiments, the intermediate addition product is a compound of formula (5), wherein the compound of formula (5) may be subsequently reacted with the second portion of the compound of formula (3), (12), or (13), or may be used as such for producing the sphingoid base of formula (1).

In some embodiments, the portion-wise addition is performed by adding the same type of compound of formula (3), (12), or (13), in each portion. Accordingly, in some embodiments, the portion-wise addition is performed by adding the same type of aldehyde, the same type of ketone, the same type of acetal, or the same type of ketal, in each portion.

In some embodiments, the portion-wise addition is performed by adding a different type of compound of formula (3), (12), or (13). Accordingly, in some embodiments, the portion-wise addition is performed by adding a different type of aldehyde, a different type of ketone, a different type of acetal, or a different type of ketal, in each portion.

In some embodiments, the portion-wise addition is performed by adding an aldehyde in the first portion and a ketone in the second portion.

In some embodiments, the portion-wise addition is performed by adding a ketone in the first portion and an aldehyde in the second portion.

In some embodiments, the portion-wise addition is performed by adding an acetal in the first portion and a ketal in the second portion.

In some embodiments, the portion-wise addition is performed by adding a ketal in the first portion and an acetal in the second portion.

In some embodiments, the portion-wise addition is performed by adding a ketone in the first portion and an acetal in the second portion.

In some embodiments, the portion-wise addition is performed by adding an acetal in the first portion and a ketone in the second portion.

Typically, the condensation of the compound of formula (2) with a compound of formula (3), (12), or (13), or a combination thereof is performed in the presence of an inorganic or an organic acid.

In some embodiments, the acid is a Broønsted acid such as hydrochloric acid, sulfuric acid, phosphoric acid, polyphosphoric acid, acetic acid, camphor sulfonic acid, p-toluene sulfonic acid, methanesulfonic acid, trifluoromethanesulfonic acid, perchloric acid, montmorillonite, zeolites, or an acidic cation exchange resin.

In some embodiments, the acid is a Lewis acid such as, aluminium(III) chloride, iron(III) chloride, zinc(II) chloride, or boron trifluoride diethyl etherate.

In some embodiments, the condensation of the compound of (2) with a compound of formula (3), (12), or (13), or a combination thereof is performed in the presence of trifluoromethanesulfonic acid.

In some embodiments, the condensation of the compound of (2) with a compound of formula (3), (12), or (13), or a combination thereof is performed in the presence of methanesulfonic acid.

In some embodiments, the condensation of the compound of formula (2) with a compound of formula (3), (12), or (13), or a combination thereof is performed in the presence of p-toluenesulfonic acid.

In some embodiments, the condensation of the compound of formula (2) with a compound of formula (3), (12), or (13), or a combination thereof is performed in the presence of boron trifluoride diethyl etherate.

The acid may be used in catalytic amounts, equimolar amounts or in excess. Typically, between about 0.01 to about 3 molar equivalents of the acid is used, based on the amount of the compound of formula (2). Preferably, between about 0.5 to about 1.5 molar equivalents of the acid are used, based on the amount of the compound of formula (2). Therefore, in a preferred embodiment, about 0.5, 0.6, 0.7, 0.8, 0.9, 1.0, 1.1, 1.2, 1.3, 1.4, or 1.5 molar equivalents of the acid are used based on the amount of the compound of formula (2). During the condensation of the compound of formula (2) with a compound of formula (3), (12), or (13), or a combination thereof water and/or an alcohol are formed as the elimination products. The person skilled in the art, would understand that when the compound of formula (2) is condensed with an aldehyde or a ketone the elimination product is water, whereas when the compound of formula (2) is condensed with an acetal or a ketal the elimination product is an alcohol. The person skilled in the art, would also understand that when a combination of a ketone and an acetal is used a mixture of an alcohol and water is formed as the elimination product.

The water and/or the alcohol formed during the condensation of the compound of formula (2) with a compound of formula (3), (12), or (13), or a combination thereof may be removed via distillation, and/or via the use of a water reacting reagents, and/or via the use of a drying agent.

In some embodiments, the removal of water and/or the alcohol formed during the condensation reaction is performed via atmospheric azeotropic distillation.

In some embodiments, the removal of the water formed during the condensation reaction is performed via the use of a water reacting reagents such as trimethyl orthoformate, or trimethyl orthoacetate.

In some embodiments, the removal of the water formed during the condensation reaction is performed via the use of a drying agent such as molecular sieves, calcium(II) chloride, magnesium sulfate, copper(II) sulfate, and sodium sulphate.

The condensation reaction is typically performed in an organic solvent such as acetonitrile, ethyl acetate, propyl acetate, butyl acetate, dichloromethane, tetrahydrofurane, 2-methyltetrahydrofurane, dioxane, xylene, methyl-tertbutyl ether, toluene, diisopropyl ether. Preferably acetonitrile, and toluene.

In some embodiments, the condensation of the compound of formula (2) with a compound of formula (3), (12), or (13), or a combination thereof is performed under kinetic control, wherein the condensation reaction is performed at a temperature between about 25° C. and about 90° C., preferably between about 50° C. and about 85° C., and wherein the reaction time is between about 1 hour to about 10 hours, preferably between about 1 hour to about 6 hours. Accordingly, in some embodiments, the condensation of the compound of (2) with a compound of formula (3), (12), or (13), or a combination thereof is performed at a temperature of about 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C. C, 79° C., 80° C., 81° C., 82° C., 83° C., 84° C., or 85° C., and a reaction time of about 1, 1.5, 2, 2.5, 3, 3.5 4, 4.5, 5, 5.5 or 6 h.

In some embodiments, the condensation of the compound of (2) with a compound of formula (3), (12), or (13), or a combination thereof is performed at a temperature of about 75° C., 76° C., 77° C., 78° C., 79° C., 80° C., 81° C., 82° C., 83° C., 84° C., or 85° C., and a reaction time of about 3 h.

In some embodiments, the condensation of the compound of formula (2) with a compound of formula (3), (12), or (13), or a combination thereof is performed at a temperature of about 75° C., 76° C., 77° C., 78° C., 79° C., 80° C., 81° C., 82° C., 83° C., 84° C., or 85° C., and a reaction time of about 3.5 h.

In some embodiments, the condensation of the compound of formula (2) with a compound of formula (3), (12), or (13), or a combination thereof is performed at a temperature of about 75° C., 76° C., 77° C., 78° C., 79° C., 80° C., 81° C., 82° C., 83° C., 84° C., or 85° C., and a reaction time of about 4 h.

In some embodiments, the condensation of the compound of formula (2) with a compound of formula (3), (12), or (13), or a combination thereof is performed at a temperature of about 75° C., 76° C., 77° C., 78° C., 79° C., 80° C., 81° C., 82° C., 83° C., 84° C., or 85° C., and a reaction time of about 4.5 h.

In some embodiments, the condensation of a compound of formula (2) with a compound of formula (3), (12), or (13), or a combination thereof is performed at a temperature of about 75° C., 76° C., 77° C., 78° C., 79° C., 80° C., 81° C., 82° C., 83° C., 84° C., or 85° C., and a reaction time of about 5 h.

In some embodiments, the condensation of the compound of formula (2) with a compound of formula (3), (12), or (13), or a combination thereof is performed at a temperature of about 75° C., 76° C., 77° C., 78° C., 79° C., 80° C., 81° C., 82° C., 83° C., 84° C., or 85° C., and a reaction time of about 5.5 h.

In some embodiments, the condensation of the compound of formula (2) with a compound of formula (3), (12), or (13), or a combination thereof is performed at a temperature of about 75° C., 76° C., 77° C., 78° C., 79° C., 80° C., 81° C., 82° C., 83° C., 84° C., or 85° C., and a reaction time of about 6 h.

In some embodiments, the condensation of the compound of formula (2) with a compound of formula (3), (12), or (13), or a combination thereof may be performed under thermodynamic control, wherein the condensation reaction is performed at a temperature between about 80° C. and about 150° C., preferably between about 80° C. and about 125° C., and wherein the reaction time is between about 10 hours to about 120 hours, preferably between about 24 hours to 120 hours. Accordingly, in some embodiments, the condensation of the compound of formula (2) with a ketone, an aldehyde, an acetal, a ketal, or a combination thereof is performed at a temperature about 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C., 122° C., 123° C., 124° C., or 125° C. and a reaction time of about 24 h, 48 h, 72 h, 96 h, or 120 h.

In some embodiments, the condensation of the compound of formula (2) with a compound of formula (3), (12), or (13), or a combination thereof is performed at a temperature of about 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., or 110° C. and a reaction time of about 24 h.

In some embodiments, the condensation of the compound of formula (2) with a compound of formula (3), (12), or (13), or a combination thereof is performed at a temperature of about 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., or 110° C. and a reaction time of about 48 h.

In some embodiments, the condensation of the compound of formula (2) with a compound of formula (3), (12), or (13), or a combination thereof is performed at a temperature of about 80° C., 81° C., 82° C., 83° C., 84° C., 85° C. 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., or 110° C. and a reaction time of about 72 h.

In some embodiments, the condensation of the compound of formula (2) with a compound of formula (3), (12), or (13), or a combination thereof is performed at a temperature of about 80° C., 81° C., 82° C., 83° C., 84° C., 85° C., 86° C., 87° C., 88° C., 89° C. 90° C., 91° C., 92° C., 93° C., 94° C., 95° C., 96° C. 97° C., 98° C., 99° C., 100° C., 101° C., 102° C., 103° C., 104° C., 105° C., 106° C., 107° C., 108° C., 109° C., or 110° C. and a reaction time of about 96 h.

In some embodiments, the condensation of the compound of formula (2) with a compound of formula (3), (12), or (13), or a combination thereof is performed at a temperature of about 80° C., 81° C., 82° C., 83° C., 84° C. 85° C., 86° C., 87° C., 88° C., 89° C., 90° C., 91° C., 92° C., 93° C., 94° C., 95° C., 96° C. 97° C., 98° C., 99° C., 100° C., 101° C., 102° C., 103° C., 104° C., 105° C., 106° C., 107° C., 108° C., 109° C., or 110° C. and a reaction time of about 120 h.

The method according to the present invention comprises one or more steps of processing the protected derivate of the compound of formula (2) to obtain the sphingoid base of formula (1). Preferably, the method of the present invention comprises the steps of:
providing a compound of formula (2);
subjecting the compound of formula (2) to a condensation reaction with a compound of formula (3), (12), or (13), or a combination thereof thereby obtaining a protected derivative of the compound of formula (2), as any of the protected derivates described herein;
introducing a leaving group at the C-4 position of the protected derivative of the compound of formula (2), wherein said protected derivative is a compound of formula (4), (5), (6), or (10), by substitution or replacement of the C-4 hydroxyl group thereby obtaining a compound of formula (14), (15), (16), or (17) respectively:

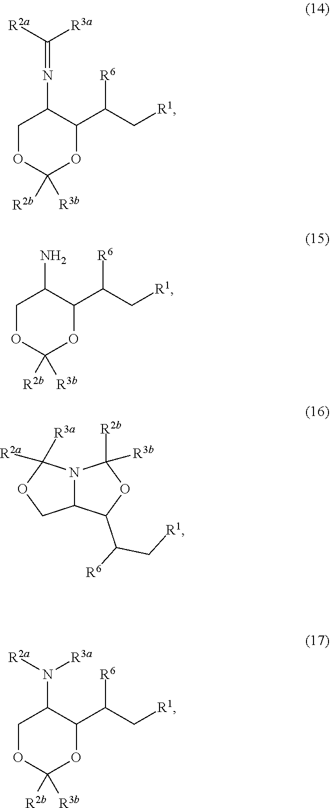

wherein
R¹, R^{2a}, R^{3a}, R^{2b}, and R^{3b} are as defined as for the compounds of formula (4), (5), (6), or (10),
R⁶ is a leaving group selected from a halide, a sulfonate, or a phosphite;
   reacting the compound of formula (14), (15), (16), or (17) with a base thereby inducing an elimination reaction, and thereby obtaining a compound of formula (18), (19), (20) or (21), respectively:

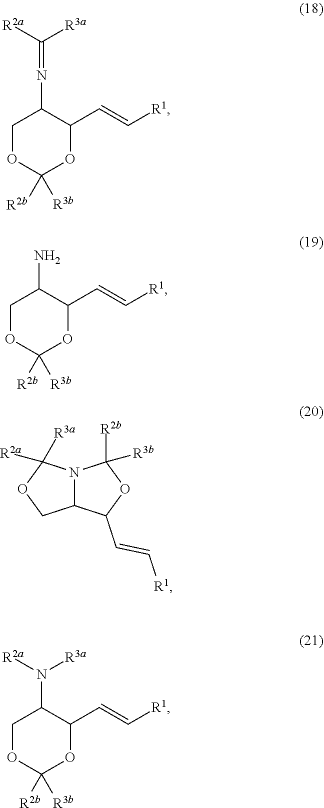

wherein
R¹, R^{2a}, R^{3a}, R^{2b}, and R^{3b} are as defined as for the compounds of formula (14), (15), (16), or (17);
   subjecting the compound of formula (18), (19), (20) or (21) to acidic treatment thereby producing a sphingoid base of formula (1), or a salt thereof.

The leaving group introduced at the C-4 position of the compound of formula (4), (5), (6), or (10), may be a halide, such as iodide, bromide, fluoride, and chloride, a sulfonate such as mesylate (—OMs), tosylate (—OTs), triflate (—OTf), nosylate (—ONs), brosilate, imidazole-1-sulfonate (—OSO₂Im), 2-methylimidazole-1-sulfonate, triazole-1-sulfonate, or dichlorophosphite (—OPCl₂). The introduction of the leaving group is typically performed under neutral or basic conditions to keep the protecting groups at the C-1, C-2, and C-3 position of the compound formula of (4), (5), (6), or (10) stable, thereby producing the compound of formula (14), (15), (16), or (17), respectively.

When the leaving group R⁶ of the compound of formula (14), (15), (16), or (17) is a halide, it may preferably be introduced via a deoxy-halogenation reaction by using a triphenylphosphine (PPh3)/imidazole reagent known by the person skilled in the art. The halide is preferably iodide or bromide, more preferably iodide. Deoxy-halogenation usually takes place in dichloromethane.

When the leaving group R⁶ of the compound of formula (14), (15), (16), or (17) is a dichlorophosphite, it may preferably be introduced by using POCl₃ and a base such as pyridine or triethyl amine in dichloromethane or toluene.

When the leaving group R⁶ of the compound of formula (14), (15), (16), or (17) is a sulfonate, it may preferably be introduced by using the corresponding sulfonic acid halide or sulfonyl azole and a base. The base is preferably an organic base, more preferably pyridine, triethylamine (TEA), or diisopropylethylamine (DIPEA). The sulfonic acid halide is preferably a sulfonic acid chloride or a sulfonic acid fluoride, more preferably p-toluenesulfonyl chloride or p-toluenesulfonyl fluoride. In some embodiments, the sulfonyl azole is preferably 1,1′-sulfonyldiimidazole or 1-tosyl-1,2,3-triazole, preferably 1,1′-sulfonyldiimidazole.

Sulfonylation may preferably be performed in ethyl acetate, propyl acetate, butyl acetate, tetrahydrofuran, 2-methyltetrahydrofurane, acetonitrile, propionitrile, dioxane, xylene, methyl-tertbutyl ether, toluene, diisopropyl ether. More preferably, the sulfolnylation is performed in acetonitrile or toluene.

The elimination reaction is induced via the treatment of the compound of formula (14), (15), (16), or (17) with a base thereby forming a double bond between the C-4 and C-5 carbon atoms, and thereby obtaining a compound of formula (18), (19), (20) or (21), respectively.

In a preferred embodiment, the elimination reaction is performed by using bases such as KOtBu, DBU, or DBN, wherein high selectivity towards the trans-double bond is achieved, and the product is obtained in high purity.

Preferably, organic solvents such as ethyl acetate, propyl acetate, butyl acetate, tetrahydrofuran, 2-methyltetrahydrofurane, acetonitrile, propionitrile, dioxane, xylene, methyl-tertbutyl ether, toluene, diisopropyl ether, or N,N-dimethylformamide may be used to perform the elimination reaction. The elimination reaction is typically performed at a temperature range between about 60° C. to about 150° C., preferably at a temperature range between about 80° C. to about 150° C. Therefore, in some embodiments the elimination reaction may be performed at a temperature of about 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C., 130° C., 131° C., 132° C., 133° C., 134° C., 135° C., 136° C., 137° C., 138° C., 139° C., 140° C., 141° C., 142° C., 143° C., 144° C., 145° C., 146° C., 147° C., 148° C., 149° C., or 150° C.

The acidic treatment is typically performed by using inorganic or organic acids such as sulfuric acid, hydrochloric acid, hydrobromic acid, phosphoric acid, polyphosphoric acid, acetic acid, camphor sulfonic acid, p-toluenesulfonic acid, methane sulfonic acid, trifluoromethanesulfonic acid, perchloric acid, aluminium(III) chloride, iron(III) chloride, zinc(II) chloride, boron trifluoride diethyl etherate, montmorillonite, zeolites, or an acidic cation exchange resin. Preferably, the acid is selected from sulfuric acid, hydrochloric acid, or p-toluenesulfonic acid.

The acid may be used in excess, or in equimolar amounts. Typically, about 1 to about 4 molar equivalents of the acid may be used, based on the amount of the compound of formula (18), (19), (20) or (21). Preferably, about 1 to about 2 molar equivalents of the acid are used, based on the amount of the compound of formula (18), (19), (20) or (21). Therefore, in a preferred embodiment, about 1.0, 1.1, 1.2, 1.3, 1.4, 1.5, 1.6, 1.7, 1.8, 1.9 or 2.0 molar equivalents of the acid are used, based on the amount of the compound of formula (18), (19), (20) or (21).

Typically, the acidic treatment is performed in an organic solvent such as acetonitrile, propionitrile, methanol, ethanol, propanol, butanol, at a temperature range of about 0 to about 100° C., preferably at a temperature range of about 0° C. to about 60° C. In some embodiments the reaction may be performed at a temperature of about 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C.

In some embodiments, the introduction of the leaving group and the elimination reaction is performed stepwise, meaning that the compound of formula (14), (15), (16), or (17) is first isolated and then subjected to the elimination reaction.

In some embodiments, the introduction of the leaving group and the elimination reaction is performed in one-pot, meaning that the compound of formula (14), (15), (16), or (17) is directly subjected to the elimination reaction without isolation.

In some preferred embodiments, for the compound of formula (14), (15), (16), (17), (18), (19), (20), and (21) R¹ is a linear unsubstituted saturated C₁₃ alkyl, one of R^{2a} and R^{3a}, and one of R^{2b} and R^{3b} is hydrogen, and the other rest is a phenyl.

In some embodiments, for the compound of formula (14), (15), (16), (17), (18), (19), (20), and (21) R¹ is a linear unsubstituted saturated C₁₃ alkyl, one of R^{2a} and R^{3a}, and one of R^{2b} and R^{3b} is hydrogen, and the other rest is p-methoxyphenyl.

In some embodiments, for the compound of formula (14), (15), (16), (17), (18), (19), (20), and (21) R¹ is a linear unsubstituted saturated C₁₃ alkyl, one of R^{2a} and R^{3a}, and one of R^{2b} and R^{3b} is hydrogen, and the other rest is a is p-methylphenyl.

In some embodiments, for the compound of formula (14), (15), (16), (17), (18), (19), (20), and (21) R¹ is a linear unsubstituted saturated C₁₃ alkyl, one of R^{2a} and R^{3a}, and one of R^{2b} and R^{3b} is hydrogen, and the other rest is a is p-chlorophenyl.

In some embodiments, for the compound of formula (14), (15), (16), (17), (18), (19), (20), and (21) R¹ is a linear unsubstituted saturated C₁₃ alkyl, one of R^{2a} and R^{3a} is hydrogen, and the other rest is phenyl, and one of R^{2b} and R^{3b} is hydrogen and the other rest a p-methoxyphenyl.

In some embodiments, for the compound of formula (14), (15), (16), (17), (18), (19), (20), and (21) R¹ is a linear unsubstituted saturated C₁₃ alkyl, one of R^{2a} and R^{3a} is hydrogen, and the other rest is p-methoxyphenyl, and one of R^{2b} and R^{3b} is hydrogen and the other rest a phenyl.

In some embodiments, for the compound of formula (14), (15), (16), (17), (18), (19), (20), and (21) R¹ is a linear unsubstituted saturated C₁₃ alkyl, and R^{2a}, R^{3a}, R^{2b} and R^{3b} are methyl.

In some embodiments, for the compound of formula (14), (15), (16), (17), (18), (19), (20), and (21) R¹ is a linear, unsubstituted, saturated C₁₃ alkyl, one of R^{2a} and R^{3a} is hydrogen, and the other rest is phenyl, and R^{2b} and R^{3b} are methyl.

The skilled person will understand that in formulas showing a specific compound, like for example formulas (1), (2), (4), (5), (6), (7), (8), (9), (10), (11), (14), (15), (16), (17), (18), (19), (20), (21), (22), (23), (24), (25), (26), and (27) unless the chemical formula expressly describes a carbon atom having a particular stereochemical configuration, the formula is intended to cover compounds where such a stereocenter has an R or an S configuration, or wherein a double bond has a cis or a trans configuration.

In some preferred embodiments, the stereochemical configuration of the C-2, C-3, and C-4 carbon atoms of compounds of formulas (2), (4), (5), (6), (7), (8), (9), (10), (11), (14), (15), (16), (17), (22), (23), (24), (25), (26), and (27) is (2S,3S,4R).

In some preferred embodiments, the compounds of formulas (2), (4), (5), (6), (7), (8), (9), (10), (11), (14), (15), (16), and (17) are compounds of formula (28), (29), (30), (31), (32), (33), (34), (35), (36), (37), (38), (39), and (40) respectively, or salts thereof:

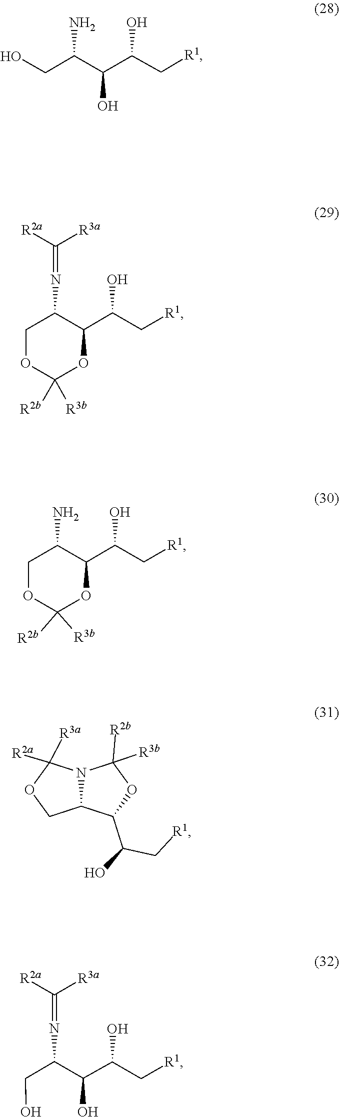

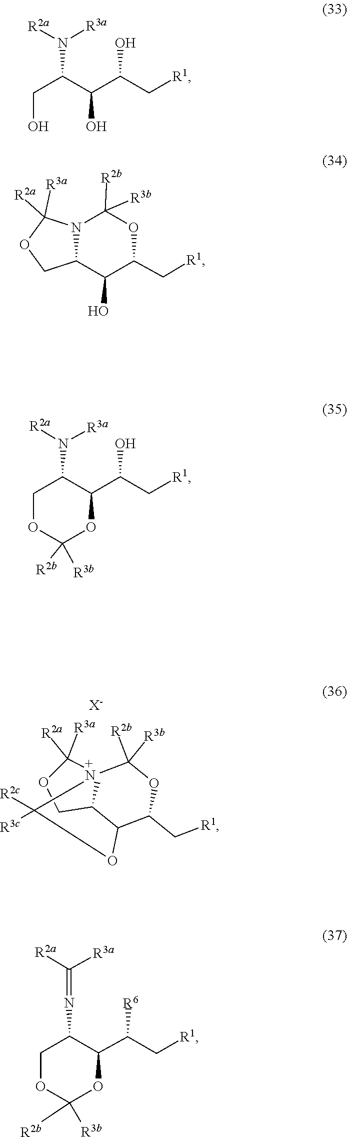

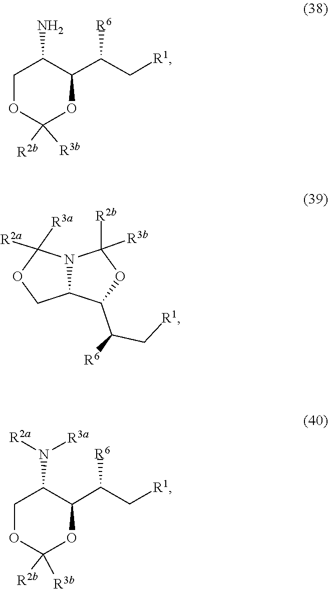

wherein
for compounds (28) to (40) R¹ is as defined as for the compound of formula (2), for compounds (29) to (40) R^{2a}, R^{3a}, R^{2b}, R^{3b}, R^{2c}, R^{3c}, R⁶, and X⁻ are as defined as for the compounds of formula (2) to (11), and as for the compounds of formula (14) to (17), and wherein preceding embodiments defining R¹, R^{2a}, R^{3a}, R^{2b}, R^{3b}, R^{2c}, R^{3c}, R⁶, and X⁻ apply also to compounds (28) to (40).

In some preferred embodiments, for the compounds of formula (29), (30), (31), and (32) R¹ is a linear unsubstituted saturated C₁₃ alkyl, one of R^{2a} and R^{3a}, and one of R^{2b} and R^{3b} is hydrogen, and the other rest is a phenyl.

In some preferred embodiments, the stereochemical configuration of the C-2, C-3, and C-4 carbon atoms of compounds of formulas (1), (18), (19), (20), and (21) is (2S,3R,4E).

In some preferred embodiments, the compounds of formulas (1), (18), (19), (20), and (21) are compounds of formula (41), (42), (43), (44), and (45), respectively:

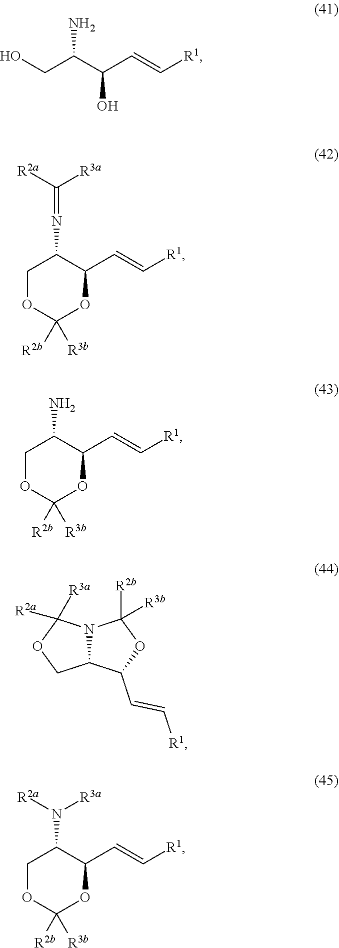

wherein
for compounds (41) to (45) R¹ is as defined as for the compound of formula (1), for compounds (42) to (45) R^{2a}, R^{3a}, R^{2b}, and R^{3b} are as defined as for the compounds of formula (18) to (21), and wherein preceding embodiments defining R¹, R^{2a}, R^{3a}, R^{2b}, and R^{3b} apply also to compounds (41) to (45).

In some preferred embodiments, for the compound of formula (42), (43), (44), and (45), R¹ is a linear, unsubstituted, saturated C₁₃ alkyl, one of R^{2a} and R^{3a}, and one of R^{2b} and R^{3b} is hydrogen, and the other rest is a phenyl.

In some embodiments, compounds of formulas (1) and (2), compounds of formulas (4) to (11), and compounds of formula (14) to (45) may be produced or utilized in the form of salts, preferably in the form of pharmaceutical acceptable salts.

In some embodiments the salts of compounds of formulas (1) and (2), the salts of compounds of formulas (4) to (11), and the salts of compounds of formulas (14) to (45) may be formed from the following acids: hydrochloric acid, hydrobromic acid, sulfuric acid, phosphoric acid, polyphosphoric acid, acetic acid, camphor sulfonic acid, p-toluene sulfonic acid, methane sulfonic acid, trifluoromethanesulfonic acid, perchloric acid.

In some embodiments the salts of compounds of formulas (1) and (2), the salts of compounds of formulas (4) to (11), and the salts of compounds of formulas (14) to (45) are formed from sulfuric acid. Accordingly, in some embodiments, the compounds of formulas (1) and (2), the compounds of formulas (4) to (11), and the compounds of formulas (14) to (45) are in the form of sulfate and/or hydrosulfate salts.

As mentioned, the present invention in one aspect provides a method for the production of a sphingoid base of formula (1), from a compound of formula (2), wherein the method comprises obtaining the compound of formula (2).

Accordingly in some embodiments, the method for the production of a sphingoid base of formula (1) from a compound of formula (2) described herein may further comprise one or more steps for the production of the compound of formula (2) that precede the step of condensation of said compound described in detail above.

In some embodiments, wherein the compound of formula (2) is D-ribo-phytosphingosine, the compound of formula (2) may be obtained in one step from commercially available acetylated analogues of D-ribo-phytosphingosine, such as for example tetraacetylphytosphingosine (TAPS), triacetylphytosphingosine (TriAPS), diacetylphytosphingosine (DiAPS) or monoacetylphytosphingosine (MAPS), or a mixture thereof, which can be purchased from established manufacturers, e.g. Merck.

Accordingly, in some embodiments, the method of the invention relates to the compound of formula (2) which is D-ribo-phytosphingosine, and the method comprises a step of obtaining D-ribo-phytosphingosine from a commercially available acetylated analogue thereof (i.e. TAPS, TriAPS, DiAPS, or MAPS, or a mixture thereof) via a step of the acid and/or base hydrolysis of said acetylated analogue preceding the step of condensation described herein.

In other embodiments, the compound of formula (2), may be obtained via a process comprising several steps, including a step of microbial fermentation, wherein the microbial fermentation yields at least one acetylated analog of the compound of formula (2), followed by one or more steps of separation of the at least one fermented acetylated analogue of the compound of formula (2) from fermentation matter (i.e. fermentation broth comprising the microbial cells), and, finally, a step of the acid and/or base hydrolysis of the at least one fermented acetylated analog to obtain the compound of formula (2).

The microbial fermentation of at least one fermented acetylated analogue of the compound of formula (2) is preferably performed using an oleaginous yeast, e.g. Pichia ciferrii, Yarrowia lipolytica, etc.

The microbial cell could be a wild type cell, i.e. a cell having a genome that is occurring in nature, that is able to naturally produce one or more acetylated analogs of the compound of formula (2), or it could be a recombinant microbial cell, i.e. a cell having a genome manipulated/engineered by man to differ from a genome that is occurring in nature, that is genetically engineered to produce one or more acetylated analogs of the compound of formula (2). By “genome” is herein understood the total genetic material comprised by the cell, i.e. chromosomal and extrachromosomal (e.g. plasmid) genetic material.

Accordingly, in one embodiment, the present invention relates to a method comprising:
fermenting at least one acetylated analog of the compound of formula (2) by a microbial cell that is capable of producing said compound;
extracting/purifying/isolating the at least one acetylated analog of the compound of formula (2) from the total fermentation material comprising said microbial cell and fermentation broth, or, optionally, from the microbial cell biomass separated from the fermentation broth;
subjecting the at least one acetylated analog of the compound of formula (2) extracted/purified/isolated from the total fermentation material to hydrolysis thereby producing the compound of formula (2);
subjecting the compound of formula (2) to a condensation reaction with a compound of formula (3) according to any of the corresponding embodiments described above, thereby obtaining a protected derivate of the compound of formula (2) as any of the protected derivates described herein;
processing the protected derivate of the compound of formula (2) according to any of the corresponding embodiments described above, thereby producing the sphingoid base of formula (1).

In some preferred embodiments, the compound of formula (2) is D-ribo-phytosphingosine, accordingly, in some preferred embodiments, the at least one acetylated analog of the compound of formula (2) is preferably selected from tetraacetylphytosphingosine (TAPS), triacetylphytosphingosine (TriAPS), diacetylphytosphingosine (DiAPS) and monoacetylphytosphingosine (MAPS), or it is a mixture of TAPS, TriAPS, DiAPS, and MAPS.

A microbial cell capable of producing at least one acetylated analog of the compound of formula (2), is preferably an oleaginous yeast. As mentioned above, both natural (wild type) and genetically modified (recombinant) cells can be employed to produce compounds of the invention. In one preferred embodiment, the invention relates to the fermentation of at least one acetylated analog of the compound of formula (2) by cells of yeast Pichia ciferrii (P. ciferrii), also known as Wickerhamomyces ciferrii (W. ciferrii). In one embodiment, the P. ciferrii cell is a wild type cell (i.e. a cell that does not contain any human-manipulated genetic material), in another embodiment, the cell is genetically modified for the purpose to be able to produce either/both higher amounts of at least one acetylated analog of the compound of formula (2), or/and a desired acetylated analog of the compound of formula (2), or/and any other purposes that, e.g., would improve the fermentation, e.g. increase cell viability, cell growth rate, etc. Non-limiting examples of genetically modified producing cells could be recombinant P. ciferrii, where the genes, SHM1 and SHM2, encoding L-serine hydroxymethyltransferases, L-serine deaminase gene CHA1, an LCB kinase gene PcLCB4, and/or gene ORM12 encoding a putative negative regulator of sphingolipid synthesis are deleted, and/or the C4-hydroxylase Syr2 and/or two serine palmitoyltransferase subunits, Lcb1 and Lcb2 overproduced. (see e.g Börgel D. et al., Metabolic Engineering 2012, 14: 412-426). In one embodiment, P. ciferrii cells producing the at least one acetylated analog of the compound of formula (2) are not genetically modified, i.e. the cells are of wild type. In a preferred embodiment, the invention relates to P. ciferrii strain F-60-10 (also known as W. ciferrii NRRL Y-1031 F-60-10). The strain is commercially available from ATCC (14092) or Westerdijk Fungal Biodiversity Institute, Culture Collection of Fungi and Yeast (CBS111).

Accordingly, the method of the invention in one embodiment comprises a step of producing at least one acetylated analog of the compound of formula (2) by P. ciferrii fermentation.

P. cifferii fermentation according to the invention may be performed using standard approaches of the technical field, e.g. as described in the prier art (see e.g. U.S. Pat. No. 5,958,742 or Börgel D. et al, 2012 (op. cit.).

The acetylated analogs of the compound of formula (2) produced by fermentation can be purified/extracted/isolated from the whole fermentation broth (fermentation slurry/fermentation material) comprising microbial cells, e.g. yeast cells (biomass) suspended in the liquid broth (fermentation broth). In some embodiments, the fermentation broth can be separated from the biomass, and the produced compound is then purified/extracted/isolated from the biomass. In other embodiments, it may be preferred to process the whole fermentation material (including both biomass and fermentation broth) to obtain the produced compounds of interest.

According to the invention, the compounds may be purified/extracted/isolated form the whole fermentation material or biomass by applying a suitable method known in the art, e.g. such as described in Breil N., et al., Molecules 2016, 21, 196; Duarte S. H., et al., Biochemical Engineering Journal 2017 125:230-237; Zainuddin F. M., et al., Microorganisms 2021, 9, 251. In one embodiment, the produced acetylated analog(s) of the compound(s) of formula (2) is(are) purified/extracted/isolated from the whole fermentation material, preferably, from the whole fermentation material that has been subjected to dewatering.

Typically, fermentation slurry has a dry matter content varying from around 15% (w/w) to around 35% (w/w). The terms “about”/“around” mean that the mentioned value can deviate up to 0.1-10%. Most of the acetylated analogs of the compounds of formula (2) produced by fermentation of natural stains of P. ciferrii are hydrophobic and typically contained within the production cells, and isolation of the produced compounds often involves the use of hydrophobic solvents. Therefore, in some embodiments it could be advantageous to reduce the water content/dewater the fermentation slurry. Accordingly, some embodiments of the method of the invention, comprise a step of dewatering of the fermentation slurry. This step can be done by drying of the fermentation slurry before proceeding to the step of purification/extraction/isolation of the fermented acetylated analog(s) of the compound(s) of formula (2).

The step of drying of the fermentation slurry may be performed using any conventional method (see e.g. op. cit.). In a preferred embodiment, the dewatering includes one or more steps of drying, wherein at least one of the drying steps includes the use of a fluidized or sub-fluidized bed dryer.

When multistep drying is used, the fermentation slurry, including biomass, is typically first spray dried. This can give a fine powder. The temperature of spray drying (air inlet temperature) is usually from about 160 to about 260° C., and/or the air outlet temperature is from about 75 to about 90° C. The biomass slurry is usually sprayed by a fast-rotating disk or a nozzle which generates small particles. The particles can then fall, under gravity, towards the bottom of a spray drying tower. Here, a fluid bed may be provided, which can use hot air to effect drying (suitably at around 90 to around 95° C.). Here, agglomeration can take place, and the particles can stick together. Following this, the agglomerated (granular) particles are subjected to drying, for example on a belt drying bed or on a sub-fluidized bed.

Another technique is to use a fluidized bed agglomeration. Here, powder can be fluidized in a gas flow. In the particle bed a fluid is sprayed with water that wets the powder and enhances the agglomeration. This combination of spraydrying in combination with a fluid bed after dryer is suited for the agglomeration of many different types of biomasses.

Drying can occur in air or under nitrogen. With fluidized and sub-fluidized bed drying, the temperature in the bed can be adjusted to pre-set values. These values can range widely, for example, from 35° to 120° C., such as 50 to 90° C., e. g. from 60 to 80° C.

At the start of the process, a fermentation slurry can have a dry matter content of below 30% (w/w). After spray drying, this can increase to from 75 to 90% (w/w), and after agglomeration can be from 90 to 95% (w/w), or more. The dried slurry particles obtained following fluidized (or sub-fluidized) bed drying treatment will typically have dry matter content at least around 70-75% (w/w). According to the invention, the dry fermentation matter comprises at least 4% (w/w) compound(s) of formula (2). Throughout the specification “% (w/w)” is meant the weight of the named substance contained in the 100 g of the named composition, e.g. 30% (w/w) dry matter in the fermentation slurry means that 100 g of the slurry contains 30 g of dry matter.

According to the invention, acetylated analogs of the compound of formula (2) are extracted from the dried fermentation matter in a subsequent process.

Extraction is preferably conducted using a hydrophobic solvent. The solvent employed will depend upon the compound to be extracted, but in particular one can mention C₁₋₁₀ alkyl esters (e.g. ethyl or butyl acetate), toluene, C₁₋₆ alcohols (e.g. methanol, propanol) and C₃₋₈ alkanes (e.g. hexane), and/or a supercritical fluid (e.g. liquid CO₂ or supercritical propane). In prior art techniques, the solvent has been typically employed directly on the microorganism in the broth. However, by performing extraction on the granules, one can significantly reduce the amount of solvent required, such as 20 to 30 times less solvent may be needed in order to perform the extraction. Not only does this result in a significant economic saving, because less solvent is used, it also minimizes emission problems. By using granules, the surface area available to the solvent can be particularly high and therefore one can obtain good yields.

In one preferred embodiment, the acetylated analogs of the compound of formula (2) are extracted using supercritical liquid CO₂ (scr CO₂) from dry granules of P. ciferrii fermentation slurry prepared by use of fluid bed spray-granulator. Previously, it was reported (US20030143659) that acetylated analogs of the compound of formula (2), in particular, tetraacetylphytosphingosine (TAPS), tend to degrade at temperatures of around 180° C. and therefore the fermented compounds cannot be efficiently recovered from dried fermentation broth prepared by multi-step drying due to high temperatures used in the procedure (e.g. for initial spray-drying of fermentation slurry/biomass). However, surprisingly, drying the P. cifirrii fermentation slurry of the present invention in a fluid bed spray-granulator (which uses a high temperature (around 180-200° C.) for initial particularization of the material by spray-drying) does not significantly reduce the total content of the fermented acetylated derivatives of the compound of formula (2) in the material. In particular, about 70-90% (w/w) of the compounds of interest obtained by fermentation can be recovered from the dried fermentation matter processed as described above.

As mentioned above, the compounds can be extracted/isolated from the dried fermentation matter by using various organic solvents, e.g. toluene or methanol, or by super critical liquid CO₂ (scrCO₂) extraction. In a preferred embodiment, the acetylated derivatives of the compound of formula (2) of the invention are extracted from the dry fermentation matter by scrCO₂ extraction.

The scrCO₂ extraction is a known technique for extraction of oils and lipids produced by microbial fermentation and the standard procedures applicable for extraction of oils or lipids produced by fermentation (see e.g. Duarte S. H., et al. Biochemical Engineering Journal 2017, 125, 230-237; Zainuddin M. F., et al Microorganisms 2021, 9, 251). Any of the described methods applicable for extraction of compounds from a dried biomass may be used for the purposes of invention. Typically for all methods described so far, before scrCO₂ extraction, the biomass is pretreated with some cell disruptive techniques, e.g. ultrasound, microwave treatment.

According to the present invention, spray-granulated fermentation material prepared as described above is preferably extracted in a supercritical fluid equipment using carbonic anhydride at high pressures (250-500 bar) and temperatures ranging from about 40 to about 120° C. The extraction process takes typically 1-6 h. The efficiency of the extraction is high, in particular roughly 80-95% (w/w) of compounds of interest are extracted from the spray-granulation material. The compounds of interest typically constitute 50-65% (w/w) of the total extract.

The scrCO₂ extract, containing the acetylated analogs of the compound of formula (2), is, according to the invention, further subjected to a acid and/or base hydrolysis before the condensation step of claim 1, e.g. as described in working Examples 15-17 below.

### EXAMPLES

Working examples below describe non-limiting embodiments of the invention and are given only to illustrate the invention.

### General Methods and Material

¹H NMR and ¹³C NMR was recorded with a Bruker Avance II (400 MHz) spectrometer. ¹H and ¹³C chemical shifts are given in ppm (δ) relative to tetramethylsilane (δ=0.00), CDCl₃ (δ=7.26, δ=77.00) as internal standard. Thin layer chromatography (TLC) was performed with silica gel TLC-plates (Merck, Silica gel, F254) with detection by UV-absorption (254 nm) where applicable and carrying (140° C.) with ammonium molybdate (25 g/L) and cerium ammonium sulfate (10 g/L) in 10% H₂SO₄. Column chromatography was performed on Silica gel 60 (220-440 mesh ASTM, Fluka).

### Example 1

### Production of (2S,3S,4R)-1,3-benzylidene-2-aminooctadecane-1,3,4-triol (46)/(47)

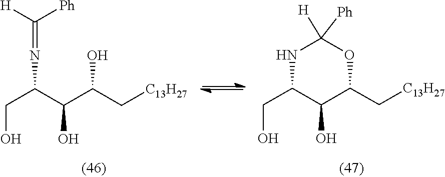

D-ribo-phytosphingosine (CAS no: 554-62-1, 5.0 g, 15.7 mmol, 1.0 eq.) was suspended in acetonitrile (25 mL), benzaldehyde (1.9 mL, 1.9 g, 18.4 mmol, 1.2 eq.) was added and the resulting suspension was stirred at ambient temperature for 22 h. The solid was filtered off, washed with acetonitrile (3×25 mL), and dried in vacuo (50 mbar/50° C./20 h) to yield: 5.6 g of compound (46)/(47) as a colorless solid (13.8 mmol, 88%, (46):(47)=3:97).

¹H-NMR (400 MHz, CDCl₃): δ (ppm)=7.56-7.46 (m, 2H), 7.40-7.28 (m, 3H), 5.18 (s, 1H), 3.85 (dd, J=10.9, 4.8 Hz, 1H), 3.78 (dd, J=10.9 Hz, 4.8 Hz, 1H), 3.47 (t, J=8.9 Hz, 1H), 3.25 (t, J=9.2 Hz, 1H), 3.00-2.85 (m, 1H), 2.32 (brₛ, 3H), 1.97-1.81 (m, 1H), 1.66-1.49 (m, 2H), 1.49-1.14 (m, 23H), 0.88 (t, J=6.6 Hz, 3H).

¹³ C-NMR (101 MHz, CDCl₃): δ (ppm)=140.0, 128.4, 126.1, 87.6, 81.0, 70.4, 63.9, 61.0, 32.2, 32.1, 29.9, 29.5, 25.4, 22.9, 14.3.

ESI-MS: calculated for [C₂₅ H₄₃NO₃⁺H]⁺: 406.3, found: 406.3.

### Example 2

### Production of Compound (48)

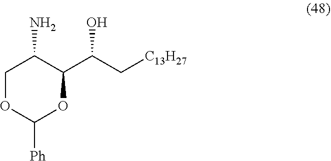

Compound (46)/(47) (5.0 g, 12.3 mmol, 1.0 eq.) was suspended in acetonitrile (15 mL), p-toluenesulfonic acid monohydrate (0.23 g, 1.23 mmol, 0.10 eq.) and benzaldehyde dimethyl acetal (2.8 mL, 2.8 g, 18.5 mmol, 1.5 eq.) were added and the mixture was stirred at reflux temperature until TLC-analysis showed complete consumption of the starting material. The reaction mixture was cooled to ambient temperature, and an aqueous solution of HCL was added to the reaction mixture. The formed solid was filtered off, washed with acetonitrile (2×5 mL) and dried in vacuo to yield compound (48) as a colorless solid (4.0 g, 9.86 mmol, yield 80%).

¹H NMR (400 MHz, CDCl₃): δ (ppm)=7.50-7.41 (m, 2H), 7.41-7.30 (m, 3H), 5.48 (s, 1H), 4.17 (dd, J=10.9, 4.9 Hz, 1H), 3.90 (ddd, J=8.0, 7.9, 2.7 Hz, 1H), 3.46 (dd, J=10.7, 10.7 Hz, 1H), 3.31 (dd, J=9.6, 7.7 Hz, 1H), 3.06 (ddd, J=10.1, 10.1, 5.0 Hz, 1H), 1.87-1.75 (m, 1H), 1.65-1.16 (m, 28H), 0.88 (t, J=6.8 Hz, 3H).

¹³C NMR (101 MHz, CDCl₃): δ (ppm)=137.9, 129.0, 128.4, 126.1, 100.9, 82.7, 75.0, 73.8, 49.6, 33.2, 32.1, 20.0, 29.9, 29.8, 29.8, 29.5, 25.0, 22.8, 14.3.

### Example 3

### Production of Compound (49)

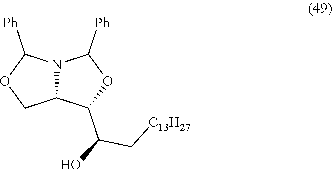

Compound (46)/(47) (5.0 g, 12.3 mmol, 1.0 eq.) and p-toluenesulfonic acid monohydrate (0.23 g, 0.12 mmol, 0.1 eq.) were suspended in acetonitrile (15 mL) and benzaldehyde dimethyl acetal (2.8 mL, 18.5 mmol, 1.5 eq.) was added. The suspension was heated to reflux until TLC-analysis showed complete consumption of the starting material. Subsequently, the solvent was removed by rotary evaporation and the residue was neutralized with triethylamine. The crude product was purified by column chromatography to yield compound (49) as colorless oil which solidified upon standing (1.4 g, yield 28%).

¹H NMR (400 MHz, CDCl₃): δ (ppm)=7.16-7.08 (m, 2H), 7.04-6.85 (m, 8H), 5.71 (s, 1H), 5.34 (s, 1H), 4.27 (dd, J=8.2, 3.6 Hz, 1H), 4.08 (d, J=8.1 Hz, 1H), 3.67-3.45 (m, 3H), 2.00-1.89 (m, 1H), 1.76-1.17 (m, 26H), 0.88 (t, J=6.8 Hz, 3H).

¹³C NMR (101 MHz, CDCl₃): δ (ppm)=142.6, 137.1, 127.6, 127.6, 127.5, 127.3, 127.2, 127.0, 89.5, 87.2, 81.5, 78.0, 67.0, 64.9, 32.3, 32.1, 29.9, 29.9, 29.8, 29.5, 25.2, 22.8, 14.3.

ESI-MS: calculated for [C₃₂H₄₇NO₃⁺H⁺]⁺: 494.4, found: 494.6.

### Example 4

### Production of Compound (50)

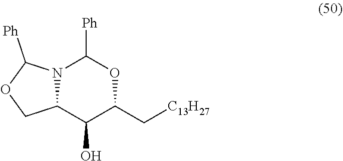

Compound (46)/(47) (5.0 g, 12.3 mmol, 1.0 eq.) and p-toluenesulfonic acid monohydrate (0.23 g, 0.12 mmol, 0.1 eq.) were suspended in acetonitrile (15 mL) and benzaldehyde dimethyl acetal (2.8 mL, 18.5 mmol, 1.5 eq.) was added. The suspension was heated at reflux until TLC-analysis showed complete consumption of the starting material. Subsequently, the solvent was removed by rotary evaporation and the residue was neutralized with triethylamine. The crude product was purified by column chromatography to yield compound (50) as colorless oil which solidified upon standing (1.80 g, yield 30%).

¹H NMR (400 MHz, CDCl₃): δ (ppm)=7.62-7.53 (m, 4H), 7.42-7.26 (m, 6H), 5.73 (s, 1H), 5.40 (s, 1H), 4.34 (dd, J=8.0, 5.0 Hz, 1H), 4.14 (dd, J=15.3, 7.7 Hz, 2H), 3.61 (ddd, J=9.7, 9.6, 5.4 Hz, 1H), 3.42-3.31 (m, 2H), 1.88-1.76 (m, 1H), 1.76-1.64 (m, 1H), 1.61 (d, J=6.1 Hz, 1H), 1.58-1.47 (m, 1H), 1.47-1.20 (m, 23H), 0.89 (t, J=6.8 Hz, 3H).

¹³C NMR (101 MHz, CDCl₃): δ (ppm) =140.0, 138.7, 129.4, 128.7, 128.6, 128.3, 127.9, 127.5, 92.5, 82.5, 73.1, 69.9, 67.9, 58.5, 32.1, 32.1, 29.9, 29.9, 29.8, 29.8, 29.5, 25.4, 22.8, 14.3.

ESI-MS: calculated for [C₃₂H₄₇NO₃⁺H⁺]⁺: 494.4, found: 494.5.

### Example 5

### Production of Compound (51)

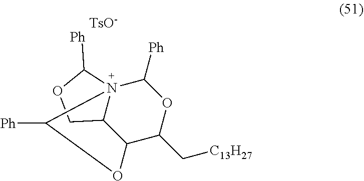

Compound (46)/(47) (1.0 eq.) and p-toluenesulfonic acid monohydrate (1 eq.) were suspended in acetonitrile (15 mL) and benzaldehyde dimethyl acetal (3 eq.) was added. The suspension was heated at reflux while continuously removing the formed alcohol by atmospheric azeotropic distillation. The reflux was continued until TLC-analysis showed complete consumption of the starting material. Subsequently, the solvent was removed by rotary evaporation and the residue was purified by column chromatography to yield compound (51) as a mixture of two isomers in a ratio of approximately 2:1.

ESI-MS: calculated for [C₃₉H₅₂NO₃⁺]⁺: 582.4, found: 582.6.

### Example 6

### Production of Compound (52)/(53)

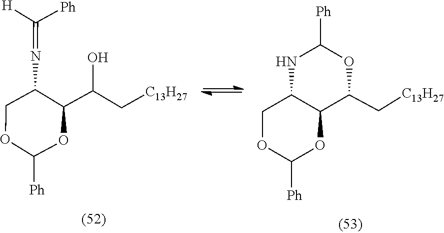

Compound (46)/(47) (5.0 g, 12.3 mmol, 1.0 eq.) was suspended in acetonitrile (15 mL), p-toluenesulfonic acid monohydrate (0.23 g, 1.23 mmol, 0.10 eq.) and benzaldehyde dimethyl acetal (2.8 mL, 2.8 g, 18.5 mmol, 1.5 eq.) were added and the mixture was stirred at reflux until TLC-analysis showed complete consumption of the starting material. The reaction mixture was cooled to ambient temperature. The formed solid was filtered off, washed with acetonitrile (2×5 mL) and dried in vacuo to yield compound (52)/(53) as a colorless solid (3.1 g, 6.22 mmol, yield 51%, (52):(53)=8:92).

¹H-NMR (400 MHz, CDCl₃): δ (ppm)=7.58-7.48 (m, 4H), 7.47-7.28 (m, 6H), 5.61 (s, 1H), 5.34 (s, 1H), 4.35 (dd, J=10.5, 4.3 Hz, 1H), 3.82 (td, J=8.4, 2.9 Hz, 1H), 3.64 (t, J=10.5 Hz, 1H), 3.36-3.22 (m, 2H), 2.00-1.86 (m, 1H), 1.69-1.52 (m, 2H), 1.53-1.39 (m, 2H), 1.40-1.17 (m, 22H), 0.89 (t, J=6.7 Hz, 3H).

¹³ C-NMR (101 MHz, CDC;₃): δ (ppm)=139.8, 137.9, 128.4, 128.4, 128.4, 126.2, 126.0, 102.0, 88.2, 81.5, 79.0, 70.2, 54.8, 32.1, 31.8, 29.9, 29.8, 29.8, 29.8, 29.8, 29.7, 29.5, 25.2, 24.5, 22.8, 14.3.

ESI-MS: calculated for [C₃₂H₄₇NO₃+H]⁺: 494.4, found: 494.4.

### Example 7

### Production of Compound (54)

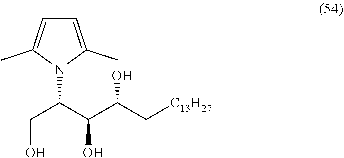

2,5-Hexanedione (2 mL) was added to a solution of d-ribo-phytosphingosine (5 g) in toluene (10 mL). The mixture was stirred at room temperature until TLC-analysis showed complete consumption of the starting materials. At this point, p-toluenesulfonic acid (300 mg) was added and the mixture was refluxed for 8 hours while continuously removing the formed water by atmospheric azeotropic distillation. The mixture was cooled to room temperature, and triethylamine (0.5 mL) and toluene (50 mL) were added. The mixture was washed with HCl (1N, 50 mL), water (50 mL) and NaHCO₃ solution (50 mL), dried, filtered, and concentrated. The crude product was purified by column chromatography to yield compound (54) as a syrup.

¹H NMR (400 MHz, CDCl₃): δ (ppm)=5.8 (s, 2 H), 4.2 (m, 3 H), 3.9 (m, 1 H), 3.5 (m, 1 H), 3.2 (s_{br}, 1 H), 2.3 (s_{br}, 6 H), 1.5-1.1 (m, 24 H), 0.9 (t_{br}, 3H).

¹³ C NMR (101 MHz, CDCl₃):δ 77.34, 77.02, 76.70, 75.52, 73.20, 63.65, 57.61, 31.93, 31.88, 30.34, 29.70, 29.69, 29.66, 29.65, 29.58, 29.46, 29.36, 25.92, 22.69, 14.10.

Compound (54) was acetylated and purified to yield compound (55).

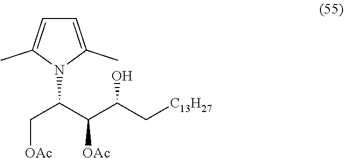

¹H NMR (400 MHz, CDCl₃): δ (ppm)=5.70 (dd, 2H), 5.62 (dd, 1H), 4.76 (dd, 1H), 4.42 (ddd, 1H), 4.34 (dd, 1H), 4.20 (dd, 1H), 2.32, 2.15, 2.05, 1.94 (s, 3H), 1.88 (s, 3H), 1.37-0.93 (m, 24H) 0.80 (t, 3H).

¹³C NMR (400 MHz, CDCl₃): δ 170.4, 170.30, 170.0, 133.2, 125.4, 109.0, 106.8, 72.9, 71.6, 63.7, 53.8, 31.9, 29.7, 29.7, 29.6, 29.6, 29.5, 29.3, 29.2, 29.0, 27.8, 25.8, 22.7, 20.9, 20.6, 14.5, 14.1, 13.1.

### Example 8

### Production of Compound (56)

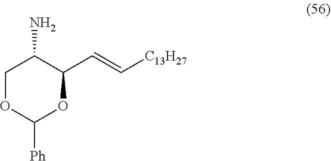

Compound (48) (1.0 g, 2,47 mmol, 1.0 eq.) and 1-(p-tolenesulfonyl)imidazole (0.67 g, 3.01 mmol, 1.3 eq.) was suspended in acetonitrile (10 mL) and 1,8-diazabicyclo[5.4.0]undec-7-ene (0.74 mL, 4.93 mmol, 2.0 eq.) was added. The reaction mixture was heated to reflux until TLC showed complete consumption of starting material. The reaction mixture was allowed to cool to room temperature, the solvent was removed by rotary evaporation and the residue was purified by flash column chromatography. Compound (56) was obtained as colorless solid (0.42 g, yield: 44%).

¹H NMR (400 MHz, CDCl₃): δ (ppm)=7.56-7.47 (m, 2H), 7.42-7.28 (m, 3H), 5.89 (ddd, J=15.4, 6.7, 6.7 Hz, 1H), 5.58-5.47 (m, 2H), 4.29 (dd, J=11.0, 5.0 Hz, 1H), 3.82 (dd, J=8.5, 8.5 Hz, 1H), 3.52 (dd, J=10.8, 10.8 Hz, 1H), 2.87 (ddd, J=10.4, 9.2, 4.9 Hz, 1H), 2.09 (tdd, J=7.4, 5.0, 1.6 Hz, 2H), 1.48-1.35 (m, 2H), 1.36-1.15 (m, 20H), 0.99 (s, 2H), 0.88 (t, J=6.7 Hz, 3H).

¹³C NMR (101 MHz, CDCl₃): δ (ppm)=138.1, 137.3, 129.0, 128.4, 126.9, 126.3, 101.3, 85.7, 72.6, 48.7, 32.6, 32.0, 29.8, 29.8, 29.8, 29.7, 29.6, 29.5, 29.4, 29.1, 22.8, 14.3.

### Example 9

### Production of Compound (57)

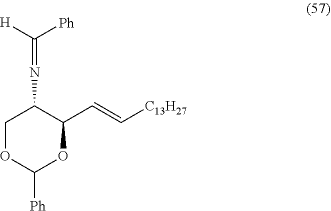

Compound (52)/(53) (2.00 g, 4.05 mmol, 1 eq.) and 1-(p-toluenesulfonyl)-imidazole (1.35 g, 6.08 mmol, 1.5 eq.) were suspended in acetonitrile (8 mL) and 1,8-diazabicyclo[5.4.0]undec-7-ene (0.91 mL, 6.08 mmol, 1.5 eq.) were added. The mixture was heated at reflux until TLC showed complete consumption of starting material. The reaction mixture was cooled to room temperature and water was added (0.4 mL). The resulting solid was filtered, cover-washed with acetonitrile/water (20:1) (3×2 mL) and dried under vacuum. Compound (57) was obtained as a colorless solid (0.92 g, yield: 48%).

¹H NMR (400 MHz, CDCl₃): δ (ppm)=8.30 (s, 1H), 7.77-7.69 (m, 2H), 7.61-7.53 (m, 2H), 7.49-7.31 (m, 6H), 5.75 (dd, J=15.1, 7.3 Hz, 1H), 5.71 (s, 1H), 5.42 (dd, J=15.5, 6.8 Hz, 1H), 4.49 (dd, J=9.0, 6.9 Hz, 1H), 4.22-4.10 (m, 2H), 3.44 (ddd, J=9.2, 9.2, 6.1 Hz, 1H), 2.07-1.87 (m, 2H), 1.36-1.04 (m, 22H), 0.89 (t, J=6.7 Hz, 3H).

¹³C NMR (101 MHz, CDCl₃): δ (ppm)=163.9, 138.3, 135.9, 135.8, 131.2, 129.0, 128.8, 128.4, 128.4, 126.7, 126.4, 101.2, 81.6, 71.2, 68.8, 32.5, 32.1, 29.8, 29.8, 29.8, 29.6, 29.6, 29.5, 29.2, 22.8, 14.3.

### Example 10

### General Procedure for the Production of D-erythro-sphingosine Sulfate

Compound (56), or compound (57) were dissolved/suspended in acetonitrile and water and 96% aq. sulfuric acid (1.2 eq.) was added. The mixture was stirred at room temperature until TLC showed complete conversion of the starting material. The reaction mixture was cooled to a temperature between 0° C. and 5° C., the resulting suspension was filtered and the solid washed with acetonitrile and dried in vacuo.

¹H NMR (400 MHz, MeOD): δ (ppm)=5.85 (dtd, J=15.1, 6.8, 1.1 Hz, 1H), 5.48 (ddt, J=15.3, 6.8, 1.5 Hz, 1H), 4.33-4.26 (m, 1H), 3.80 (dd, J=11.6, 4.0 Hz, 1H), 3.67 (dd, J=11.7, 8.3 Hz, 1H), 3.21 (ddd, J=8.5, 4.4, 4.4 Hz, 1H), 2.14-2.06 (m, 2H), 1.51-1.10 (m, 27H), 0.92 (t, J=6.8 Hz, 3H).

¹³C NMR (101 MHz, MeOD): δ (ppm)=136.5, 128.5, 59.4, 58.5, 33.4, 33.1, 30.8, 30.8, 30.7, 30.6, 30.5, 30.4, 30.2, 23.7, 14.4.

### Example 11

### Preparation of D-erythro-sphingosine Sulfate Starting from Compound (56)

Compound (56) (0.5 g, 1.23 mmol, 1.00 eq.) was subjected to the general procedure of example 7. D-erythro-Sphingosine sulfate was obtained as colorless solid (0.46 g, 94%).

### Example 12

### Preparation of D-erythro-sphingosine Sulfate Starting from Compound (57)

Compound (57) (1.00 g, 2.1 mmol, 1 eq.) was subjected to the general procedure of example 7. D-erythro-Sphingosine sulfate was obtained as colorless solid (0.81 g, yield: 97%).

### Example 13

### Fermentation of Acetylated Analogs of Sphingoid Bases and Downstream Processing of the Fermentation Material

Sphingoid bases (phytosphingosine) in the acetylated forms (TAPS—Tetraacetylphytosphingosine, TriAPS—Triacetylphytosphingosine, DiAPS—Diacetylphytosphingosine and MAPS—Monoacetylphytosphingosine) were produced by growing wild type P. ciferrii strain F-60-10 (ATCC 14092) according to the following procedure:

A seed culture for the fed-batch culture was grown by adding 1% of a strain to a 250 ml baffled shake flask containing 100 ml of the culture medium also used for the selection procedure. The flasks were incubated at 30° C. and 250 rpm.

For the actual TAPS production, a Jupiter 2 fermenter (Solaris) with a working volume of 1.5-1.7 was used. Stirring was done by three 6-blade Rushton-type impellers. pH and dissolved oxygen were measured by InPro electrodes. Airflow was regulated at a set flow rate. The vessel with electrodes was sterilised (autoclaved) at 120° C. for 20 min prior to inoculation with the seed culture. pH was controlled by addition of a 12.5% ammonium hydroxide solution.

The culture medium composition (conc. in g/L):

| | |
|---|---|
| 7.50 | NH₄H₂PO₄ |
| 7.50 | KH₂PO₄ |
| 25.00 | Glycerol |
| 1.32 | NaCl |
| 0.46 | Trace mineral solution (Fe (0.9%), Cu (0.1%), Zn (0.35%), |
| | I (0.04%), Mn (0.07%), B (0.04%), Mo (0.1%)) |
| 1.32 | MgSO₄•7H₂O |
| 2.26 | Vitamin solution (C (0.1%), B1 (0.2%), B3 (0.2%), B5 (0.2%), |
| | B6 (0.03%), B7(0.002%), B12 (0.13%)) |

The culture conditions:

| Dissolved O₂ | 0-40% | |
|---|---|---|
| Temperature | 28-34° | C. |
| pH | 5.5-8.5 | |
| Stirring rate | 400-1900 | rpm |
| Air flow | 0.5-1.0 | vvm |
| Antifoam | 0.3 | ml/l |

Inoculation of the fermenter was done with 10% (v/v) of the seed culture. When glycerol in the batch phase was depleted (as indicated by the sudden and marked increase in DO (typically after a period of 20-25 hours), a feed solution (glycerol 75% w/v/MgSO₄.7H₂O 2 g/L) was gradually fed into the fermenter in steps over 24 h.

The total amount of feed (500-850 g glycerol) was added over a period of approximately 90 hours. Total biomass and sphingoid base concentration were regularly monitored in samples taken from the fermenter which were analyzed as described below:

Twice a day a sample is taken and checked for Wet Cell Mass—WCM (g/kg), OD₆₀₀, TAPS titre (g/l), MAPS titre (g/l) and sterility. The pH should be checked once a day. The pH in the sample should be measured immediately, since it can change quickly.

WCM control: 0.3 ml of 10 ml of weighted sample to be centrifuged and the pellet and supernatant to be weighted. The WCM expressed in g/kg is calculated by =(weight of pellet/(weight of supernatant×1000.

TAPS titer control: a sample of the broth to be mixed with a methanol:acetonitrile solution and stirred for 10 min. Afterwards, the sample is centrifuged and the supernatant is analyzed by HPLC.

MAPS titer control: a sample of the broth to be mixed with a methanol:acetonitrile solution and a base. Afterwards, the sample is centrifuged, and the supernatant is analyzed by HPLC.

Typically, maximum biomass levels of 250-550 g/l were reached for a total feed of 1100-1800 g/l of glycerol, yielding a concentration of total sphingoid bases of at least 10-14 g/l after 110-140 hours of fermentation.

Following fermentation, the whole fermentation broth/fermentation slurry were collected and dried using an industrial fluid bed spray-granulator that uses the intense particle mixing in the fluid bed. The slurry to be processed was filtered from waste particles and sprayed via binary nozzles into the granulator. The inlet temperature was around 200° C. The dried droplets of the fermentation slurry, having size of around 30-70 micrometer were immersed in the fluid bed where they were granulated (due outer covering with a layer of liquid) and further dried. Typical size of particles of the fermentation material obtained by the process was around 0.5 mm to 2.0 mm. The particle moisture content was about 1.1% to 4.5%.

### Example 14

### Extraction of Acetylated Derivatives of D-ribo-phytosphingosine from Spray-Granulated Fermentation Material

14.1. Direct Extraction with Toluene

Spray-granulated material was extracted with toluene over 30-60 min at a temperature ranging from room temperature to reflux temperature. Typically, 2-3 extractions are required to extract about 70-80% (w/w) of the overall compounds of interest (acetylated D-ribo-phytosphingosine derivatives TAPS, TriAPS, DiAPS, MAPS) present in the spray-granulated material. After filtering and concentration of the solvent a final clear orange/brownish oil material enriched in the components of interest 40-55% (w/w) was obtained.

14.2 Soxhlet Extraction with Toluene

The extraction was done similar to Example 14.1, but using a Soxhlet like extraction apparatus, and performing the extraction with refluxing toluene. Extraction was performed for about 2-8 h. Soxhlet extraction with toluene resulted in similar extraction yields and composition of the final extract as those of the direct solvent extraction.

14.3 Direct Extraction with Methanol

Spray-granulated material was extracted with methanol over 30-60 min at a temperature ranging from room temperature to reflux temperature. Preferably the extraction was carried out at a temperature around the methanol boiling point. Typically, 2-3 extractions are required to extract around 95% (w/w) of the overall compounds of interest (acetylated phytosphingosine derivatives namely: tetraacetylphytosphingosine (TAPS), triacetylphytosphingosine (TriAPS), diacetylphytosphingosine (DiAPS) and monoacetylphytosphingosine (MAPS)) present in the spray-granulated material. After filtering and concentration, a final orange/brownish oil/wax enriched in the components of interest containing roughly 10-20% (w/w) was obtained.

14.4 Soxhlet Extraction with Methanol

The extraction is performed similarly to Example 14.3, but using a Soxhlet like extraction apparatus, and performing the extraction with refluxing methanol. Extraction was performed over 2-6 h. Soxhlet extraction with methanol resulted in similar extraction yields and composition of the final extract as those of the direct solvent extraction.

14.5 Supercritical Fluid Extraction with CO₂

Spray-granulated material was extracted in a supercritical fluid equipment using carbonic anhydride at high pressures (250-500 bar) and temperatures ranging from about 40-120° C. The extraction was performed for about 1-6 h. About 80-95% (w/w) of the compounds of interest is extracted from the spray-granulation material. The orange/brownish oil/wax obtained from this process, typically contains 50-65% of components of interest (a mixture of TAPS, TriAPS, DiAPS and MAPS).

A weight ratio of TAPS:TriAPS:DiAPS:MAPS in the oil/wax material obtained by extraction of the spray-granulated fermentation matter according to Examples 14.1-5 may vary, typically the extracted material would comprise TAPS 40-60% , TriAPS 20-30% DiAPS 5-15% and MAPS 1-3% (w/w).

### Example 15

### Synthesis N-acetyl-D-ribo-phytosphingosine (MAPS) from Poly-Acetylated Phytosphingosine Analogs Contained in the Extracts of Example 14

15.1 Hydrolysis with an Inorganic Base

The oil/wax of Examples 14.1-5 was dissolved in 3-6 volumes of methanol. Sodium hydroxide (2-4 equivalents), or similar strong inorganic base and, optionally, about 1/10 volume of purified water, were added. The reaction mixture was heated at a temperature of about 50-60° C. until the reaction was completed (checked by TLC and confirmed by HPLC). The reaction mixture was then cooled to room temperature and washed with an apolar/immiscible solvent, preferably a C₆-C₇ alkane or mixture of these, for at least 3 times. The lower methanolic layer was concentrated to about 50-60% of the original volume and 4 volumes of an anti-solvent were added (typically acetonitrile).

The precipitate formed was aged for 1-12 h at room temperature and filtered. The off-white solid obtained was washed twice with cold anti-solvent and dried under vacuum at a temperature of about 40-50° C. for 8-16 h. Molar yields of 70-85% of MAPS were attained from the oil extract Alternatively, MAPS could be prepared by the same process as described above, wherein water was added (up to 10% of the total volume) during the MAPS crystallization stage.

15.2 Hydrolysis with an Organic Base

The oil/wax of Examples 14.1-5 was dissolved in 3-6 volumes of methanol. Methanolic ammonia (7M, 10-24 equivalents) and, optionally, about 1/10 volume of purified water, were added. The reaction mixture was heated at a temperature of about 40-50° C. until the reaction was completed (checked by TLC and confirmed by HPLC). The reaction mixture was then cooled to room temperature and washed with a mixture of ethyl acetate/heptane (1:1) and acetonitrile. The precipitate formed was aged for 1-12 h at room temperature and filtered. The off-white solid obtained was washed twice with cold anti-solvent and dried under vacuum at 40-50° C. for 8-16 h. Molar yields of 75-90% of MAPS were obtained from the oil extract.

Alternatively, sodium methoxide can be used as organic base. The product thus obtained (MAPS) is isolated in about the same yields and shows similar quality profile.

### 15.3 Hydrolysis Under Acidic Conditions

Alternatively, inorganic acids (e.g. hydrochloric acid) or organic acids (e.g. acetic acid) can be used to hydrolyze the acetylated phytosphingosine derivatives to MAPS. MAPS can be isolated performing minor adjustments in the isolation procedure. The quality and yields of MAPS prepared by this method are similar to the above described.

### Example 16

### Synthesis of D-ribo-phytosphingosine from N-acetyl-D-ribo-phytosphingosine (MAPS)

MAPS obtained according to Example 15 was suspended in 3-6 volumes of isobutyl alcohol. Sodium hydroxide (or an equivalent inorganic base, 3 eq.) was added to the suspension and the mixture was heated to refluxed temperature until full conversion of the starting material (checked by TLC and confirmed by HPLC). Then, the reaction mixture was cooled and washed with purified water and brine. The washed organic layer was concentrated to about 50-60% of its original volume and diluted with the same volume of acetonitrile. The suspension was heated to reflux until a clear solution formed, and then cooled slowly to room temperature until a precipitate formed, optionally stirring may be applied during cooling. The off-white solid was then washed with cold acetonitrile and dried under vacuum at 40-50° C. D-ribo-Phytosphingosine was obtained in about 70-85% yield. Alternatively, the reaction may be performed in a mixture of 1:1 of isobutyl alcohol and water. The final product D-ribo-phytosphingosine is isolated with the same yields and purity.

### Example 17

### Synthesis of D-ribo-phytosphingosine from a Mixture of Acetylated D-ribo-phytosphingosine Derivatives Obtained by Extraction of the Spray-Dried Fermentation Material According to Example 14

The oil/wax extract obtained as described in Example 14, was taken up in about 3-6 volumes of isobutyl alcohol. About 4-6 equivalents of sodium hydroxide (or a similarly strong inorganic base) were added. The reaction mixture was heated to reflux temperature until completion (checked by TLC and confirmed by HPLC).

The reaction mixture was then cooled to room temperature and washed with water and brine. The solvent was removed under vacuum, and 3-4 volumes of methanol were added to the residue. The suspension was heated until the solid was dissolved, cooled to room temperature again, and then washed with an apolar/immiscible solvent, preferably an C₆-C₇ alkane or mixture of these, for at least 3 times. The lower methanolic layer was concentrated to about 30-40% of the original volume and the same volume of an antisolvent (typically acetonitrile) was added. The precipitate formed during the latter step was left at room temperature for at least 2 h, and then filtered. The obtained off-white solid was washed twice with cold acetonitrile and dried under vacuum at 40-50° C. for 8-16 h. D-ribo-Phytosphingosine was obtained in about 40-50% yield.

1. Method for producing a sphingoid base of formula (1): 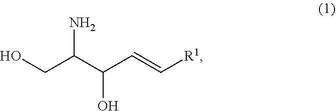
or a salt thereof,
from a compound of formula (2) 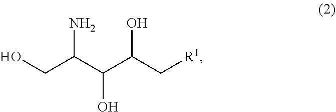
or a salt thereof,
wherein
R¹ is hydrogen, a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of an alkoxy group, a secondary, or tertiary amine, a thioether, an acyloxy group, an acylamido group, a phosphorus containing functional group, a carboxyl group, or a carbonyl group,
comprising a step of subjecting the compound of formula (2) to a condensation reaction with a compound of formula (3): 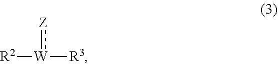
wherein
the bond [symbol] represents a double or a single bond,
W is C, or C(OR⁴),
Z is O, or OR⁵,
provided that:
when W is C, the bond [symbol] is a double bond and Z is O, or
when W is C(OR⁴), the bond [symbol] is a single bond and Z is OR⁵, and
wherein
R² and R³ are independently selected from a saturated or unsaturated C₁₋₆ alkyl, a saturated or unsaturated cycloalkyl, or an aryl, each of which may be substituted or unsubstituted, or wherein one of R² and R³ is hydrogen, and the other rest is a saturated or unsaturated C₁₋₆ alkyl, a saturated or unsaturated cycloalkyl, or an aryl, each of which may be substituted or unsubstituted, or wherein R² and R³ may form a cyclic structure,
R⁴ and R⁵ are independently selected from a C₁₋₆ alkyl, a cycloalkyl, or an aryl, each of which may be substituted or unsubstituted.

2. The method according to claim 1, wherein the condensation reaction results in the formation of at least one protected derivative of the compound of formula (2), and wherein said protected derivative is selected from the group consisting of compounds of formulas (4) to (11), or salts thereof: 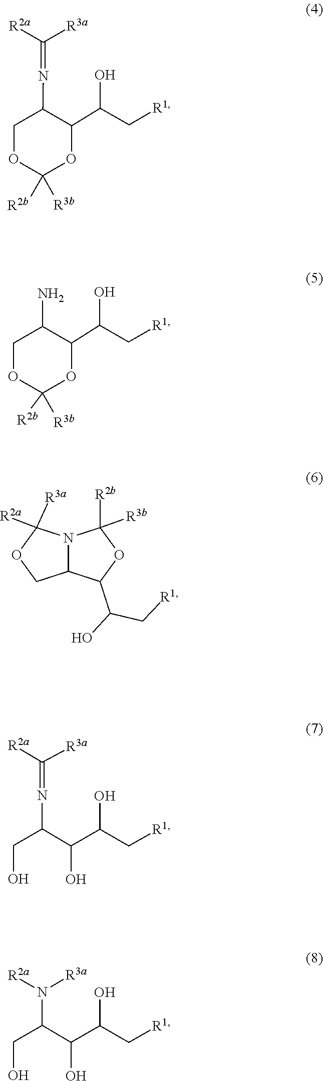 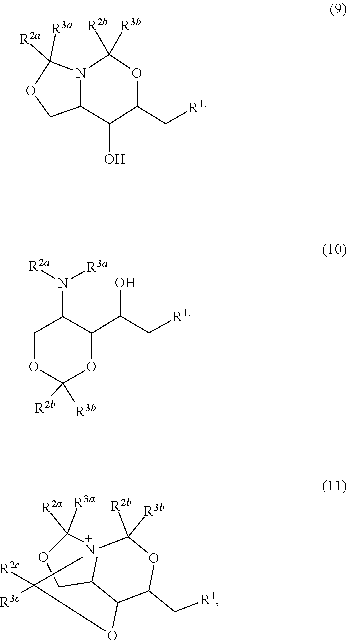
wherein
R¹, is as defined as for the compound of formula (2),
R^{2a}, R^{3a}, R^{2b}, R^{3b}, R^{2c} and R^{3c} are independently selected from a saturated or unsaturated C₁₋₆ alkyl, a saturated or unsaturated cycloalkyl, or an aryl, each of which may be substituted or unsubstituted, or wherein one of R^{2a} and R^{3a}, and/or one of R^{2b} and R^{3b}, and/or one of R^{2c} and R^{3c} is hydrogen and the other rest is a saturated or unsaturated C₁₋₆ alkyl, a saturated or unsaturated cycloalkyl, or an aryl, each of which may be substituted or unsubstituted, or wherein R^{2a} and R^{3a}, and/or R^{2b} and R^{3b}, and/or R^{2c} and R^{3c} may form a cyclic structure,
X⁻ is an organic or inorganic anion selected from chloride, perchlorate, sulfate, phosphate, polyphosphate, carboxylate, camphorsulfonate, or sulfonate.

3. The method according to claims 2, wherein the at least one protected derivative of the compound of (2) is selected from the group consisting of compounds of formulas (4), (5), (6), or (10), or salts thereof.

4. The method according to claims 2 or 3, wherein the at least one protected derivative of the compound of (2) is selected from the group consisting of compounds of formulas (4), (5), or (6), or salts thereof.

5. The method according to any one of claims 2 to 4, wherein the protected derivative of the compound of (2) is a compound of formula (4), or a salt thereof.

6. The method according to any one of claims 1 to 5, wherein the compound of formula (3) is a compound of formula (12), or (13), or a combination thereof: 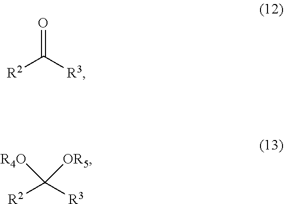
wherein, R², R³, R⁴ and R⁵ are as defined as for the compound of formula (3).

7. The method according to any one of claims 1 to 6, wherein R² and R³ of the compounds of formulas (3), (12), and (13) are independently selected from a saturated or unsaturated C₁₋₆ alkyl, a saturated or unsaturated cycloalkyl, or an aryl, each of which may be substituted or unsubstituted, or wherein R² and R³ may form a cyclic structure.

8. The method according to claim 7, wherein R² and R³ of the compounds of formulas (3), (12), and (13), are independently selected from methyl, ethyl, propyl, isopropyl, butyl, isobutyl, pentyl, isopentyl, hexyl, phenyl, or wherein R² and R³ form a cyclopentyl, or a cyclohexyl.

9. The method according to any one of claims 1 to 6, wherein for the compounds of formulas (3), (12), and (13) one of R² and R³ is hydrogen, and the other rest is a saturated or unsaturated C₁₋₆ alkyl, a saturated or unsaturated cycloalkyl, or an aryl, each of which may be substituted or unsubstituted.

10. The method according to claim 9, wherein for the compounds of formulas (3), (12), and (13) one of R² and R³ is hydrogen, and the other rest is selected from methyl, ethyl, propyl, isopropyl, butyl, isobutyl, pentyl, isopentyl, phenyl, o-methoxyphenyl, m-methoxyphenyl, p-methoxyphenyl, o-methylphenyl, m-methylphenyl, p-methylphenyl, o-chlorophenyl, m-chlorophenyl, p-chlorophenyl, o-nitrophenyl, m-nitrophenyl, p-nitrophenyl, o-trifluoromethyl-phenyl, m-trifluoromethyl-phenyl, or p-trifluoromethyl-phenyl.

11. The method according to claims 9 or 10, wherein for the compounds of formulas (3), (12), and (13) one of R² and R³ is hydrogen, and the other rest is selected from phenyl, p-methoxyphenyl, p-methylphenyl, or p-chlorophenyl.

12. The method according to any one of claims 1 to 11, wherein R⁴ and R⁵ of the compounds of formulas (3), and (13) are independently selected from methyl, ethyl, propyl, isopropyl, butyl, isobutyl, pentyl, isopentyl, preferably from methyl.

13. The method according to any one of claims 1 to 12, wherein the condensation of the compound of formula (2) with a compound of formula (3), (12), or (13), or the combination thereof further comprises the use of an acid.

14. The method according to claim 13, wherein about 0.01 to about 3 molar equivalents of the acid is used, based on the amount of the compound of formula (2).

15. The method according to claims 13 or 14, wherein the acid is a Brønsted acid, and wherein the Brønsted acid is preferably selected from hydrochloric acid, sulfuric acid, phosphoric acid, polyphosphoric acid, acetic acid, camphor sulfonic acid, p-toluene sulfonic acid, methanesulfonic acid, trifluoromethanesulfonic acid, perchloric acid, montmorillonite, zeolites, or an acidic cation exchange resin.

16. The method according to claims 13 or 14, wherein the acid is a Lewis acid, and wherein the Lewis acid is preferably selected from aluminium(III) chloride, iron(III) chloride, zinc(II) chloride, or boron trifluoride diethyl etherate.

17. The method according to any one of claims 1 to 16, wherein the condensation of the compound of formula (2) with the compound of formula (3), (12), or (13), or the combination thereof further comprises the use of a solvent.

18. The method according to claim 17, wherein the solvent is an aprotic solvent, and wherein the aprotic solvent is preferably selected from acetonitrile, ethyl acetate, propyl acetate, butyl acetate, dichloromethane, tetrahydrofurane, 2-methyltetrahydrofurane, dioxane, xylene, methyl-tertbutyl ether, toluene, diisopropyl ether.

19. The method according to any one of claims 1 to 18, wherein the condensation of the compound of formula (2) with the compound of formula (3), (12), or (13), or the combination thereof is performed at a temperature between about 25° C. and about 90° C., preferably between about 50° C. and about 85° C., and wherein the reaction time is between about 1 hour to about 10 hours, preferably between about 1 hour to about 6 hours.

20. The method according to any one of claims 1 to 18, wherein the condensation of the compound of formula (2) with the compound of formula (3), (12), or (13), or the combination thereof is performed at a temperature between about 80° C. and about 150° C., preferably between about 80° C. and about 125° C., and wherein the reaction time is between about 10 hours to about 120 hours, preferably between about 24 hours to about 120 hours.

21. The method according to any one of claims 1 to 20, wherein the molar ratio of the compound of formula (3), (12), or (13), or the combination thereof to the compound of formula (2) is from about 1 to about 4.

22. The method according to any one of claims 1 to 21, wherein the condensation of the compound of formula (2) with a compound of formula (3), (12), or (13), or a combination thereof generates water and/or an alcohol, and wherein the method further comprises a step of removing the water and/or the alcohol formed thereof.

23. The method according to claim 22, wherein the removal of the water and/or the alcohol is performed via distillation, preferably via atmospheric azeotropic distillation.

24. The method according to claim 22 wherein the removal of the water is performed using a water reacting reagent, preferably wherein said reagent is an orthoester selected from trimethyl orthoformate, or trimethyl orthoacetate.

25. The method according to claim 22, wherein the removal of the water is performed using a drying agent, preferably wherein the drying agent is selected from molecular sieves, calcium(II) chloride, magnesium sulfate, copper(II) sulfate, and sodium sulphate.

26. The method according to any one of claims 2 to 25, further comprising a step of introducing a leaving group at the C-4 position of the compound of formula (4), (5), (6), or (10) by substitution or replacement of the C-4 hydroxyl group, thereby obtaining a compound of formula (14), (15), (16), or (17) respectively, or salts thereof: 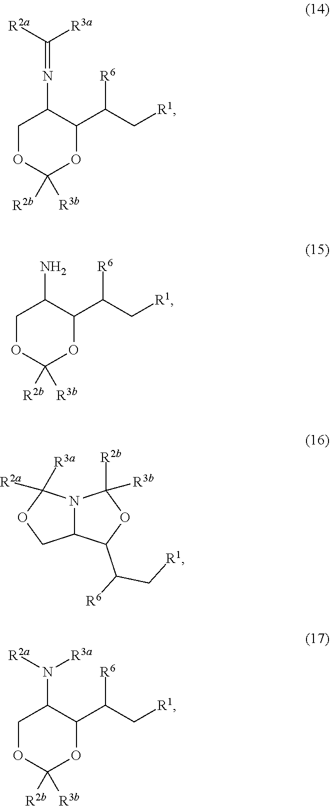
wherein
R¹, R^{2a}, R^{3a}, R^{2b}, and R^{3b} are as defined as for the compounds of formula (4), (5), (6), or (10),
R⁶ is a leaving group selected from a halide, a sulfonate, or a phosphite.

27. The method according to any one of claims 2 to 26, further comprising the step of introducing a leaving group at the C-4 position of the compound of formula (4), (5), or (6) by substitution or replacement of the C-4 hydroxyl group, thereby obtaining a compound of formula (14), (15), or (16), respectively, or salts thereof.

28. The method according to any one of claims 2 to 27, further comprising the step of introducing a leaving group at the C-4 position of the compound of formula (4) by substitution or replacement of the C-4 hydroxyl group, thereby obtaining a compound of formula (14), or a salt thereof.

29. The method according to any one of claims 26 to 28, wherein R⁶ is a halide selected from iodide, bromide, and chloride.

30. The method according to any one of claims 26 to 28, wherein R⁶ is selected from mesylate (—OMs), tosylate (—OTs), triflate (—OTf), nosylate (—ONs), brosilate, imidazole-1-sulfonate (—OSO₂Im), 2-methylimidazole-1-sulfonate, or a phosphite such as dichlorophosphite (—O₂PCl₂).

31. The method according to any one of claims 26 to 30, further comprising a step of reacting the compound of formula (14), (15), (16), or (17) with a base thereby inducing an elimination reaction, and thereby obtaining a compound of formula (18), (19), (20) or (21), respectively, or salts thereof: 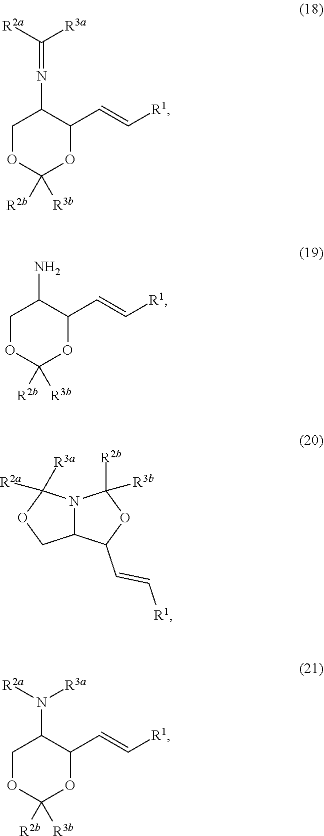
wherein
R¹, R^{2a}, R^{3a}, R^{2b}, and R^{3b} are as defined as for the compounds of formula (14), (15), (16), or (17).

32. The method according to any one of claims 26 to 31, further comprising a step of reacting the compound of formula (14), (15), or (16) with a base thereby inducing an elimination reaction, and thereby obtaining a compound of formula (18), (19), or (20), respectively, or salts thereof.

33. The method according to any one of claims 26 to 32, further comprising a step of reacting the compound of formula (14) with a base thereby inducing an elimination reaction, and thereby obtaining a compound of formula (18), or a salt thereof.

34. The method according to any one of claims 31 to 33, further comprising a step of subjecting a compound of formula (18), (19), (20) or (21) to acidic treatment thereby producing a sphingoid base of formula (1), or a salt thereof.

35. The method according to any one of claims 31 to 34, further comprising a step of subjecting a compound of formula (18), (19), or (20) to acidic treatment thereby producing a sphingoid base of formula (1), or a salt thereof.

36. The method according to any one of claims 31 to 35, further comprising a step of subjecting a compound of formula (18) to acidic treatment thereby producing a sphingoid base of formula (1), or a salt thereof.

37. The method according to any one of claims 26 to 36, further comprising the use of a solvent, wherein the solvent is an aprotic solvent, preferably selected from the group consisting of ethyl acetate, propyl acetate, butyl acetate, tetrahydrofuran, 2-methyltetrahydrofurane, acetonitrile, propionitrile, dioxane, xylene, methyl-tertbutyl ether, toluene, diisopropyl ether.

38. The method according to any one of claims 1 to 37, wherein the stereochemical configuration of the C-2, C-3, and C-4 carbon atoms of the compound of formula (2), of the compounds of formulas (4), (5), (6), (7), (8), (9), (10), and (11), and of the compounds of formulas (14), (15), (16), and (17), is (2S,3S,4R), and wherein the stereochemical configuration of the C-2, C-3, and C-4 carbon atoms the compound of formula (1), and of the compounds of formulas (18), (19), (20) and (21) is (2S,3R,4E).

39. The method according to any one of claims 1 to 38, wherein for the sphingoid base of formula (1), for the compound of formula (2), for the compounds of formulas (4), (5), (6), (7), (8), (9), (10), and (11), for compounds of formulas (14), (15), (16), and (17), and for the compounds of formulas (18), (19), (20), and (21) R¹ is a linear saturated unsubstituted C₁₃ alkyl.

40. The method according to any one of claims 2 to 39, wherein for the compounds of formulas (4), (5), (6), (7), (8), (9), (10), and (11), for the compounds of formulas (14), (15), (16), and (17), and for the compounds of formulas (18), (19), (20), and (21) one of R^{2a} and R^{3a} and one of R^{2b} and R^{3b} is hydrogen, and the other rest is a substituted or unsubstituted aryl, preferably phenyl, p-methoxy phenyl, p-chlorophenyl and p-methylphenyl.

41. The method according to any one of claims 1 to 40, wherein the sphingoid base of formula (1) is D-erythro-sphingosine.

42. The method according to any one of claims 1 to 41, wherein the compound of formula (2) is D-ribo-phytosphingosine.

43. The method according to any of claims 1 to 42, wherein the method further comprising steps of obtaining a compound of formula (2).

44. The method according to claim 43, wherein the compound of formula (2) is obtained via the steps of:
fermenting at least one acetylated analog of the compound of formula (2) in a microorganism, preferably in a yeast cell;
separating the at least one acetylated analog of the compound of formula (2) from the whole fermentation material or the microbial biomass by using an organic solvent or super-critical liquid CO₂ extraction;
subjecting the at least one acetylated analog of the compound of formula (2) to hydrolysis thereby producing the compound of formula (2);

45. The method according to any one of claims 1 to 44, wherein the method comprises the steps of:
fermenting at least one acetylated analog of the compound of formula (2) in a microorganism, preferably in a yeast cell;
separating the at least one acetylated analog of the compound of formula (2) from the whole fermentation material or the microbial biomass by using an organic solvent or super-critical CO₂ extraction;
subjecting the at least one acetylated analog of the compound of formula (2) to hydrolysis thereby producing the compound of formula (2);
subjecting the compound of formula (2) to a condensation reaction with a compound of formula (3), thereby obtaining a protected derivate of the compound of formula (2) according to any one of claims 1 to 25;
processing the protected derivate of the compound of formula (2) according to any of claims 26 to 42, thereby producing the sphingoid base of formula (1).

46. The method according to claims 44 or 45, wherein the at least one acetylated analog of the compound of formula (2) is selected from tetraacetylphytosphingosine (TAPS), triacetylphytosphingosine (TriAPS), diacetylphytosphingosine (DiAPS) and monoacetylphytosphingosine (MAPS), or a mixture thereof.

47. A protected derivative of a compound of formula (2) selected from the group consisting of compounds of formulas (4) to (11), or salts thereof: 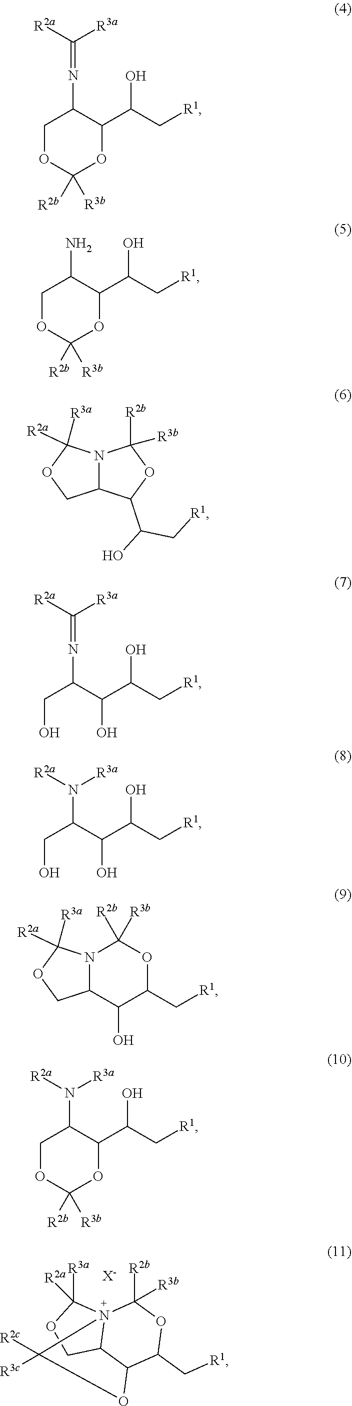
wherein
R¹ is hydrogen, a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of an alkoxy group, a secondary, or tertiary amine, a thioether, an acyloxy group, an acylamido group, a phosphorus containing functional group, a carboxyl group, or a carbonyl group,
R^{2a}, R^{3a}, R^{2b}, R^{3b}, R^{2c} and R^{3c} are independently selected from a saturated or unsaturated C₁₋₆ alkyl, a saturated or unsaturated cycloalkyl, or an aryl, each of which may be substituted or unsubstituted, or wherein one of R^{2a} and R^{3a}, and/or one of R^{2b} and R^{3b} , and/or one of R^{2c} and R^{3c} is hydrogen and the other rest is a saturated or unsaturated C₁₋₆ alkyl, a saturated or unsaturated cycloalkyl, or an aryl, each of which may be substituted or unsubstituted, or wherein R^{2a} and R^{3a}, and/or R^{2b} and R^{3b} , and/or R^{2c} and R^{3c} may form a cyclic structure,
X⁻ is an organic or inorganic anion selected from chloride, perchlorate, sulfate, phosphate, polyphosphate, carboxylate, camphorsulfonate, or sulfonate.

48. The protected derivative of the compound of formula (2) according to claim 47, wherein said protected derivative is selected from the group consisting of compounds of formulas (4), (5), (6), or (10), or salts thereof.

49. The protected derivative of the compound of formula (2) according to claim 47, wherein said protected derivative is selected from the group consisting of compounds of formulas (4), (5), or (6), or salts thereof.

50. The protected derivative of the compound of formula (2) according to claims 47, wherein said protected derivative is a compound of formula (4), or a salt thereof.

51. The protected derivative of the compound of formula (2) according to any one of claims 47 to 50, wherein the stereochemical configuration of the C-2, C-3, and C-4 carbon atoms of compounds of formulas (4), (5), (6), (7), (8), (9), (10) or (11) is (2S,3S,4R).

52. The protected derivative of the compound of formula (2) according to any one of claims 47 to 51, wherein R¹ is a linear saturated unsubstituted C₁₃ alkyl.

53. The protected derivative of the compound of formula (2) according to any one of claims 47 to 52, wherein one of R^{2a} and R^{3a} and one of R^{2b} and R^{3b} is hydrogen, and the other rest is a substituted or unsubstituted aryl, preferably phenyl, p-methoxy phenyl, p-chlorophenyl and p-methylphenyl.

54. A compound selected from the group consisting of compounds of formulas (18), (19), (20), or (21): 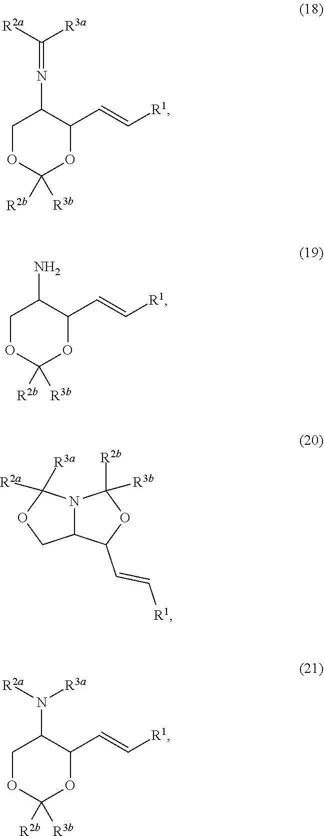
wherein
R¹ is hydrogen, a C₁₋₅₀ alkyl, preferably a C₁₋₁₅ alkyl, more preferably a C₁₀₋₁₅ alkyl, which may be saturated or contain one or more double and/or triple bonds, and/or which may contain one or more functional groups, the functional group being preferably selected from the group consisting of an alkoxy group, a secondary, or tertiary amine, a thioether, an acyloxy group, an acylamido group, a phosphorus containing functional group, a carboxyl group, or a carbonyl group,
R²a , R^{3a}, R^{2b}, and R^{3b} are independently selected from a saturated or unsaturated C₁₋₆ alkyl, a saturated or unsaturated cycloalkyl, or an aryl, each of which may be substituted or unsubstituted, or wherein one of R^{2a} and R^{3a}, and/or one of R^{2b} and R^{3b} is hydrogen and the other rest is a saturated or unsaturated C₁₋₆ alkyl, a saturated or unsaturated cycloalkyl, or an aryl, each of which may be substituted or unsubstituted, or wherein R^{2a} and R^{3a}, and/or R^{2b} and R^{3b} may form a cyclic structure.

55. The compound according to claim 54, wherein the configuration of the C-2, C-3, and C-4 carbon atoms of the compounds of formulas (18), (19), (20), and (21) is (2S,3R,4E).

56. The compound according to claims 54 or 55, wherein R¹ is a linear saturated unsubstituted C₁₃ alkyl.

57. The compound according to any one of claims 54 or 56, wherein one of R^{2a} and R^{3a} and one of R^{2b} and R^{3b} is hydrogen, and the other rest is a substituted or unsubstituted aryl, preferably phenyl, p-methoxy phenyl, p-chlorophenyl and p-methylpheny.
